(19) United States
(12) Patent Application Publication
(10) Pub. No.: US 2025/0003933 A1
(43) Pub. Date: Jan. 2, 2025

(54) IN-LINE MINI-ENVIRONMENT CONTAMINATION DETECTION AND MONITORING SYSTEM

(71) Applicant: Tricorntech Corporation, New Taipei City (TW)

(72) Inventors: Tsung-Kuan A. Chou, San Jose, CA (US); Li-Peng Wang, Taipei City (TW); Ching-Lin Hsiao, New Taipei City (TW); Wei-Shao Tung, New Taipei City (TW)

(21) Appl. No.: 18/756,650

(22) Filed: Jun. 27, 2024

Related U.S. Application Data
(60) Provisional application No. 63/511,419, filed on Jun. 30, 2023.

(51) Int. Cl. G01N 33/00

(52) U.S. Cl. CPC G01N 33/0016; G01N 33/0068; G01N 33/0073

(57) ABSTRACT

Embodiments are disclosed of an active flow control system including one or more inlets and one or more outlets. A first outlet is configured to be fluidly coupled to an inlet of a mini-environment, and a first is configured to be coupled to an outlet of the mini-environment. The active flow control system includes one or more flow control configurations; each flow control configuration corresponds to a flow control mode. The one or more flow configurations include a configuration corresponding to a sampling mode. The sampling mode includes injecting a neutral fluid from the first outlet into the inlet of the mini-environment, and directing a mixed fluid exiting through the outlet of the mini-environment to the first inlet of the active flow control system, the mixed fluid being a combination of the neutral fluid and the fluid that was in the mini-environment before injecting the neutral fluid into the mini-environment.

### CROSS-REFERENCE TO RELATED APPLICATIONS

This application claims priority under 35 U.S.C. § 119 (e) to U.S. Provisional Application No. 63/511,419, filed 30 Jun. 2023, the entire contents of which are incorporated herein by reference.

### TECHNICAL FIELD

The disclosed embodiments relate generally to airborne contamination monitoring and in particular, but not exclusively, to an apparatus, system, and method for in-line detection and monitoring of airborne contamination in stationary and mobile mini-environments.

### BACKGROUND

Air quality and airborne molecular contamination (AMC) have become increasingly important in the semiconductor, memory, and other similar high-tech industries (e.g., displays) as their processes advance. In these and other industries, AMC has been identified as a major contributor to fabrication failure rate, and the effects of AMC on manufacturing process yield get worse as the fabrication process nodes becomes smaller.

Manufacturers have been putting significant effort into on-site monitoring and into controlling facility ambient cleanliness using on-site or off-line AMC monitoring equipment. Detailed studies and ongoing improvements have been implemented to identify contamination sources and preventive procedures to reduce AMC in a facility's ambient air. But although significant efforts have been made to control facility ambient air quality, the cleanliness inside mini-environments such as process equipment modules and movable carriers (e.g., Front Opening Unified Pods (FOUPs) used as substrate/wafer transport containers in the semiconductor industry) are not well-studied.

Contamination, in particular AMC contamination, inevitably exists inside a FOUP during the process; it can come from specific process equipment/modules or from other mini-environments. Because in most situations the process equipment and substrates are enclosed in their own mini-environment, on-site facility ambient monitoring cannot detect related problems associated with process equipment/module or substrate containers such as a FOUP. When a process equipment/module or a FOUP is contaminated, cross-contamination and AMC can spread over the fabrication line, with the FOUP serving as a contamination carrier that transmits the AMC to multiple locations.

In most situations the process equipment modules and substrates have their own enclosed micro-environments, meaning that on-site facility ambient monitoring cannot capture AMC-related problems associated with process equipment modules or movable carriers. Furthermore, when one process equipment module or one movable carrier is contaminated, AMC cross-contamination can occur over a fabrication line, with the movable carrier serving as an AMC carrier that spreads contamination to various locations. As a result, it becomes extremely difficult to trace the source of contamination, even if AMC is later found in one movable carrier or process equipment module.

### BRIEF DESCRIPTION OF THE DRAWINGS

Non-limiting and non-exhaustive embodiments of the present invention are described with reference to the following figures, wherein like reference numerals refer to like parts throughout the various views unless otherwise specified.

FIGS. 1A-1B are diagrams of a process module or process equipment with a front-opening unified pod (FOUP) and an embodiment of a process by which a mini-environment such as a FOUP is purged when loaded onto the process equipment.

FIGS. 2A-2B are diagrams of embodiments of off-line sampling and analysis methods for detecting airborne molecular contamination in a mini-environment such as a FOUP.

FIGS. 3A-3B are diagrams of embodiments of in-line contamination monitoring systems for mini-environments.

FIGS. 4A-4E are diagrams of further embodiments of in-line AMC monitoring systems for mini-environments.

FIGS. 5A-5C are graphs of embodiments of flow profiles that can be applied to the modes used by the contamination monitoring system.

FIG. 6 is a block diagram of an embodiment of an in-line contamination analyzer.

FIG. 7A is a diagram of an embodiments of a load-port-based in-line contamination monitoring system for mobile mini-environments.

FIG. 7B is a pair of diagrams embodiments of a traditional load-port purge system and a load-port based in-line contamination monitoring system for mobile mini-environments.

FIGS. 8A-8D are process diagrams illustrating embodiments of the interaction between different elements of a load-port based in-line contamination monitoring system for mobile mini-environments.

FIG. 9 is a diagram illustrating an embodiment of the interaction between a contamination analyzer and multiple load-port units in a load-port based in-line contamination monitoring system for mobile mini-environments.

FIGS. 10A-10C are block diagrams of embodiments of load-port units.

FIGS. 11A-11B are block diagrams of embodiments of active flow control systems that can be used in a load-port based in-line contamination monitoring system for mobile mini-environments.

FIGS. 12A-12B are block diagrams of embodiments of a check-mode flow path for the active flow control systems shown in FIGS. 11A-11B, respectively.

FIGS. 13A-13B are block diagrams of embodiments of a sampling-mode flow path for the active flow control systems shown in FIGS. 11A-11B, respectively.

FIGS. 13C-13D are graphs of embodiments of the results of contamination measurements obtained as a result of the sampling shown in FIGS. 13A-13B, and a graph of a flow profile applied during the sampling shown in FIGS. 13A-13B.

FIGS. 14A-14B are block diagrams of embodiments of a purge-mode flow path for the active flow control systems shown in FIGS. 11A-11B, respectively.

FIGS. 15A-15B are block diagrams of embodiments of a clean-mode flow path for the active flow control systems shown in FIGS. 11A-11B, respectively.

FIG. 16 is a block diagram of an integrated load-port unit.

FIG. 17 is a diagram of a load-port based in-line contamination monitoring system for mobile mini-environments.

### DETAILED DESCRIPTION

Embodiments are described of an apparatus, system, and method for in-line monitoring of environmental conditions, including contamination, in mini-environments. Specific details are described to provide an understanding of the embodiments, but one skilled in the relevant art will recognize that the invention can be practiced without one or more of the described details or with other methods, components, materials, etc. In some instances, well-known structures, materials, or operations are not shown or described in detail but are nonetheless encompassed within the scope of the invention.

Reference throughout this specification to “one embodiment” or “an embodiment” means that a described feature, structure, or characteristic can be included in at least one described embodiment, so that appearances of “in one embodiment” or “in an embodiment” do not necessarily all refer to the same embodiment. Furthermore, the particular features, structures, or characteristics may be combined in any suitable manner in one or more embodiments.

Embodiments are described below of a load-port based in-line contamination monitoring system for mini-environments. Existing load port systems have a single function: they are used to purge mini-environments, for cleaning purposes, by providing a high-volume purge of a neutral fluid such as clean dry air (CDA) or nitrogen (N₂) to clean the mini-environment by pushing out contaminants. Existing load-port systems provide no monitoring capability to assess the contamination within the mini-environment being purged.

The described embodiments are of an in-line mini-environment contamination monitoring systems including next-generation flow control systems and analyzer arrays. In addition to the purge function performed by existing load-port systems, the disclosed measurement and monitoring systems enable monitoring of the level of contaminants inside a mini-environment. They provide several additional capabilities, including a sampling capability to sample (i.e., withdraw/extract) contaminants from inside mini-environments; an analysis capability using a combination of analyzers for detection of contaminants and general environmental conditions (e.g., temperature and humidity) in a mini-environment; a self-check capability by which the system can periodically, or by programmed action, self-check the residue of contaminants within the system is below control limits; and a self-clean capability in which, to reduce contaminant residues within the system, the system can periodically or by programmed action self-clean the whole system.

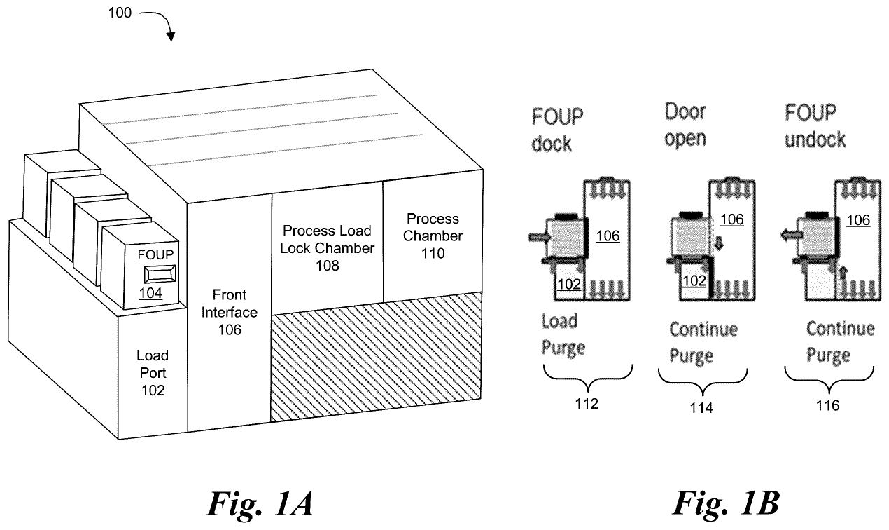

FIGS. 1A-1B together illustrate an embodiment of a process equipment module 100 and an embodiment of a purging operation for a mini-environment used with the process equipment module. In the illustrated embodiment, process equipment module 100 includes a load port 102 on which one or more mini-environments 104 (movable wafer carriers such as FOUPs in this embodiment) can be loaded; the illustrated embodiment shows a load port capable of receiving four FOUPs 104, but in other embodiments the load port can receive a different number of FOUPs than shown. FOUPs are only one possible mini-environment; other mini-environments are possible in other embodiments, as explained below. Process equipment module 100 includes three chambers in addition to load port 102: a front interface 106 next to load port 102, a process load lock chamber 108 next to front interface 106 and, finally, a process chamber 110 next to load lock chamber 108. Process chamber 110 is where the relevant manufacturing steps are performed on the items being carried in FOUP 104.

FIG. 1B illustrates an embodiment of a purging operation used by process equipment module 100. At step 112, movable carriers/FOUPs 104 are docked to load port 102 and their bottom air inlets and outlets become fluidly coupled to mating inlets/outlets in the load port. A load port unit (LPU) housed under the FOUPs in load port 102 will then purge a neutral gas such as nitrogen (N₂) or clean dry air (CDA) into the FOUP to flush air and/or contamination out from the FOUP's interior to an exhaust outlet under the load port, thereby keeping the mini-environment inside the FOUP at a required cleanliness level. At step 114, the purge continues as the FOUP's front door is opened and the wafers inside are transferred into the other chambers of the process equipment module—front interface 106, load lock chamber 108, and process chamber 110—for the required fabrication steps. Purging continues while the require fabrication steps are performed, until at step 116 the wafers are returned to the interior of the FOUP, the FOUP's door is closed, and the FOUP is undocked from the load port. The CDA purge stops after the FOUP is undocked and moved to the next process equipment module. Direct monitoring of the cleanliness inside mini-environments like the FOUP and process equipment/modules and is not well-developed.

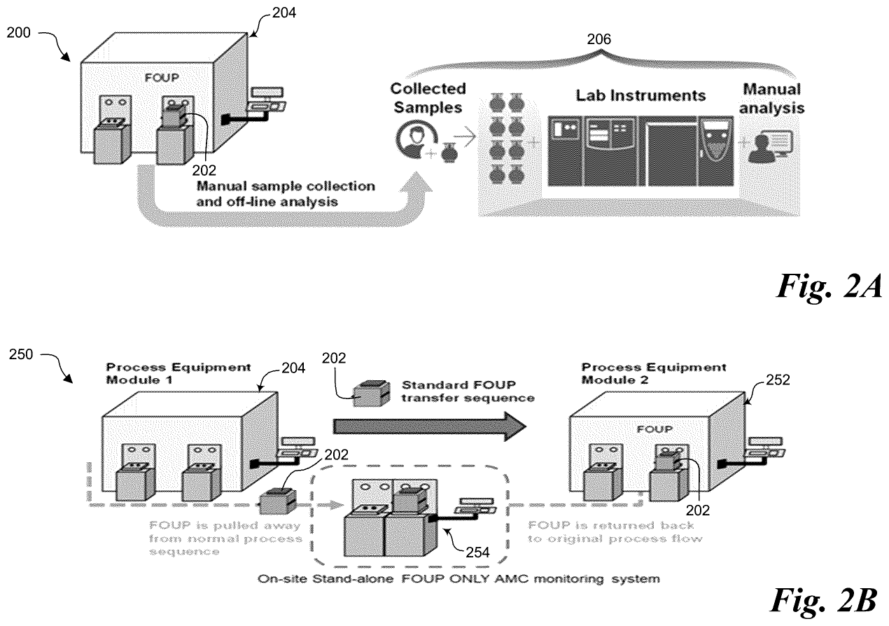

FIGS. 2A-2B illustrate embodiments of off-line sampling and measurement of the environment inside a mini-environment such as a FOUP. FIG. 2A illustrates a method that is both off-line (also known as off-process) and off-site, FIG. 2B illustrates a method that is off-line but on-site. FIG. 2A illustrates an embodiment of a current process 200 to measure and understand contamination and other conditions in the mini-environment inside the FOUP. In this method, a sample is manually collected from FOUP 202 when it is positioned on the load port of process equipment 204. The collected sample is then taken to an offsite lab 206, where the sample's chemical composition, and other environmental conditions such as temperature and humidity, can be analyzed using lab instruments. Such an approach is time consuming and can only obtain limited data from a few samples, which usually cannot capture problematic FOUPs—i.e., FOUPs whose interior environments do not meet specifications—in time. As a result, tracing contamination back to its source is hardly feasible.

FIG. 2B illustrates another embodiment of a process 250 that uses an on-site contamination analysis system—e.g., an on-site FOUP-only AMC analysis system. In process 250, when the wafers are finished in process equipment 204 and returned to a corresponding mini-environment such as FOUP 202, the normal process sequence would require that FOUP 202 be taken directly to process equipment 252 for the next manufacturing step, as shown at the top of the figure. But in this process the FOUP is instead pulled out of its normal process sequence and sent to an on-site FOUP environmental measurement, analysis, and monitoring system 254 for analysis of the air inside the FOUP.

Although contamination analysis system 254 can be an on-site monitoring tool, it is an off-process monitoring method because FOUP 202 no longer follows its regular process sequence. Because the FOUP must be pulled away from its normal fabrication sequence, this process introduces additional uncertainty due to interference in the normal process sequence. Also, in such an approach contamination analysis system 254 has limited capacity, meaning it can only screen a limited number of FOUPs and can thus significantly impact the process throughput. Even if contamination analysis system 254 can increase its screening capacity, it still adds extra steps to the fabrication, and that can further reduce process throughput. Therefore, such an approach is limited to screening FOUPs. It does not serve the purpose of in-line measurement and monitoring of the mini-environment inside the FOUPs.

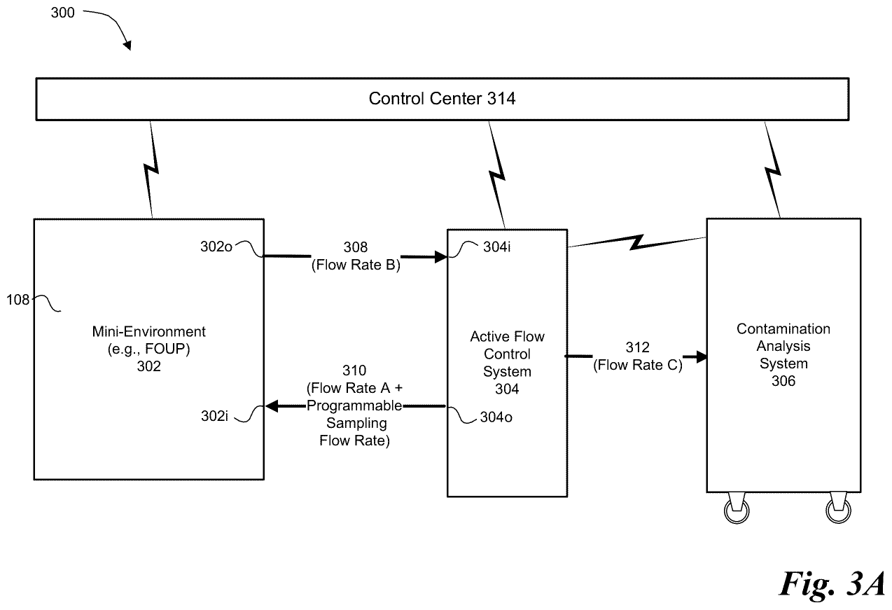

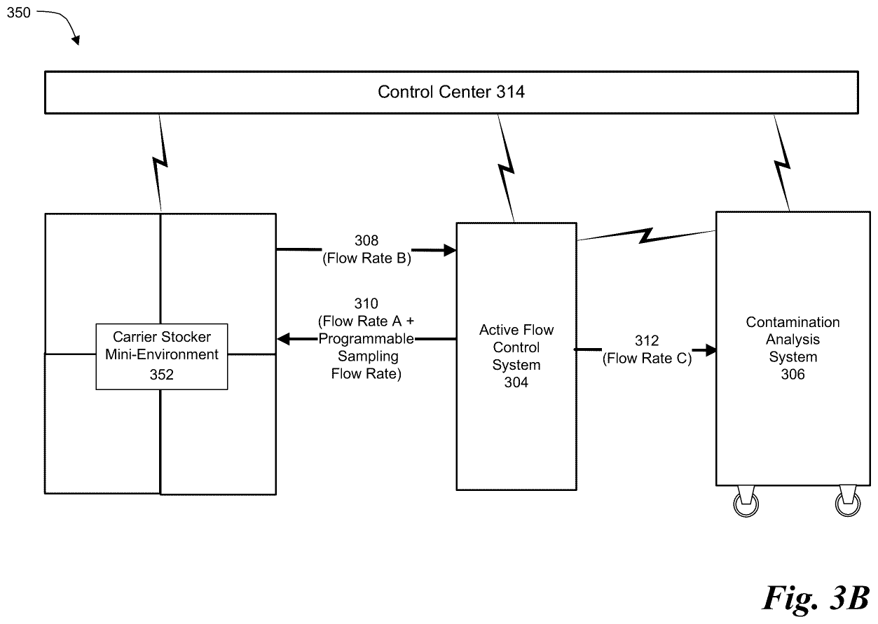

FIGS. 3A-3B illustrate embodiments of systems and methods for in-line measurement and monitoring in mini-environments. FIG. 3A illustrates an embodiment of a system and method 300 for sampling and analyzing the environment inside a mini-environment 302. System 300 includes an active flow control system 304, a contamination analysis system 306, and a control center 314, all of which work together to analyze and monitor the environment inside a mini-environment 302. In the illustrated embodiment mini-environment 302 is a mobile carrier such as a FOUP, but other embodiments can use other types of mini-environments (see, e.g., FIG. 3B).

Mini-environment 302 includes a fluid inlet 302i and a fluid outlet 3020. The illustrated embodiment of mini-environment 302 has one fluid inlet and one fluid outlet, but other embodiments can have multiple inlets, multiple outlets, or both, and in still other embodiments the number of inlets need not be the same as the number of outlets. Mini-environment 302 is fluidly coupled to active flow control system 304, so that mini-environment 302 and flow control system 304 can exchange fluids: inlet 302i is coupled by fluid line 310 to an outlet of active flow control system 304, and outlet 3020 is coupled by fluid line 308 to an inlet of active flow control system 304.

Active flow control 304 is a load-port unit (LPU) fluidly coupled to mini-environment 302, so that mini-environment 302 and flow control system 304 can exchange fluids: inlet 302i is coupled by fluid line 310 to outlet 3040, and outlet 3020 is coupled by fluid line 308 to inlet 304i. Active flow control system 304 is also fluidly coupled by fluid line 312 to contamination analysis system 306.

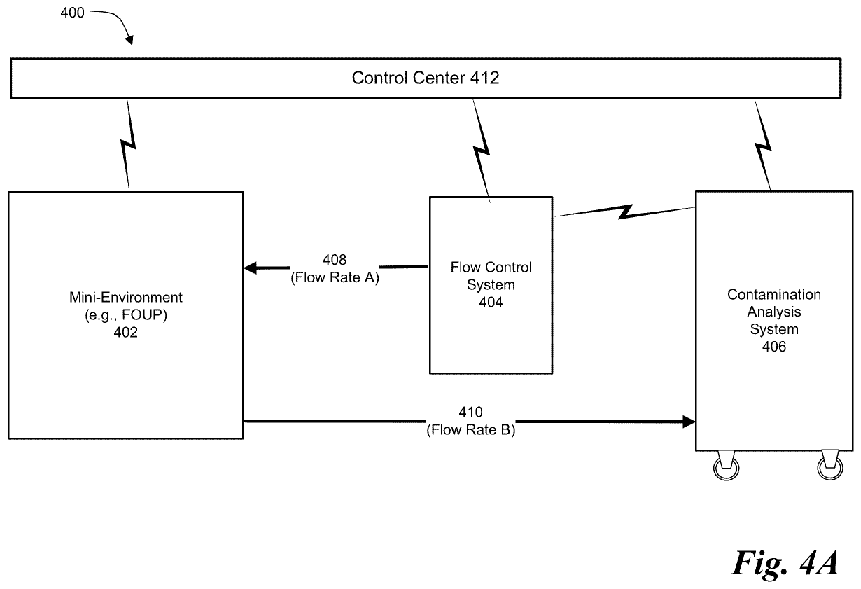

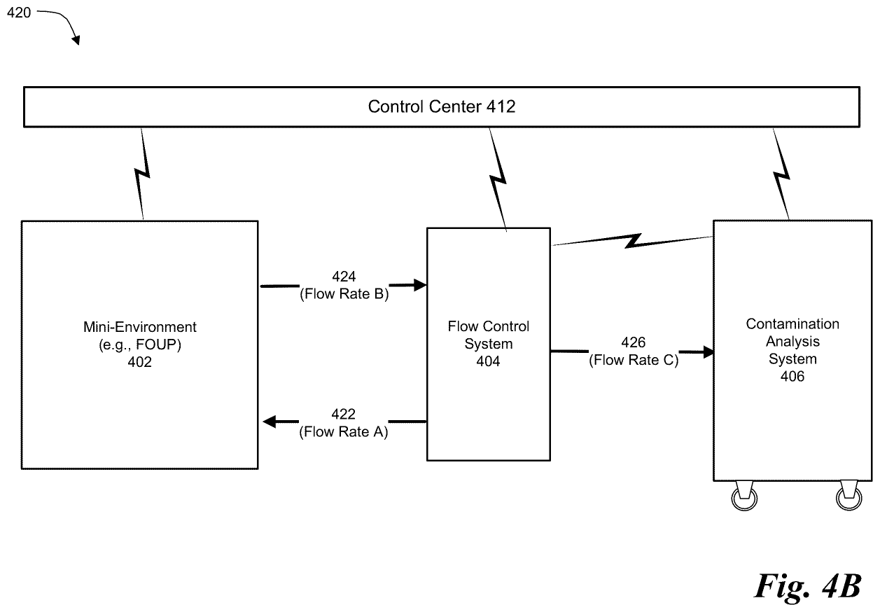

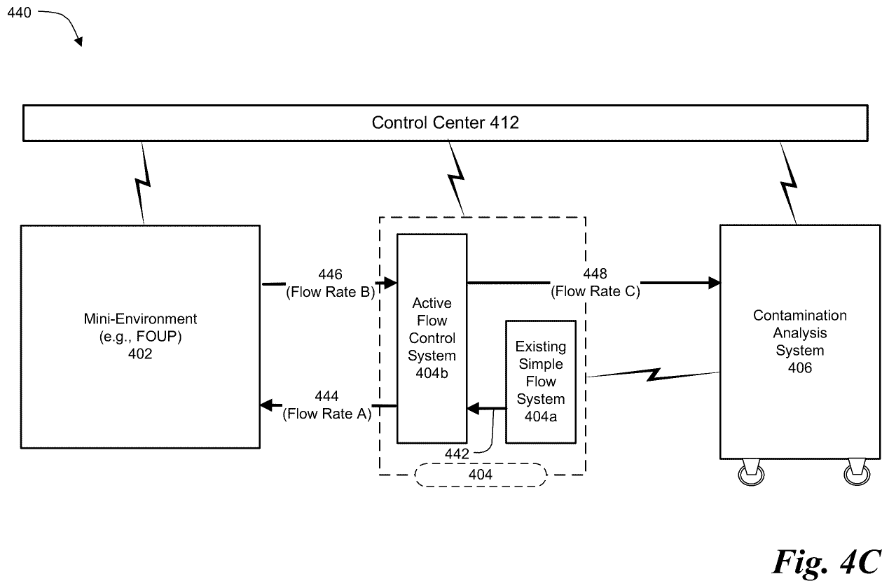

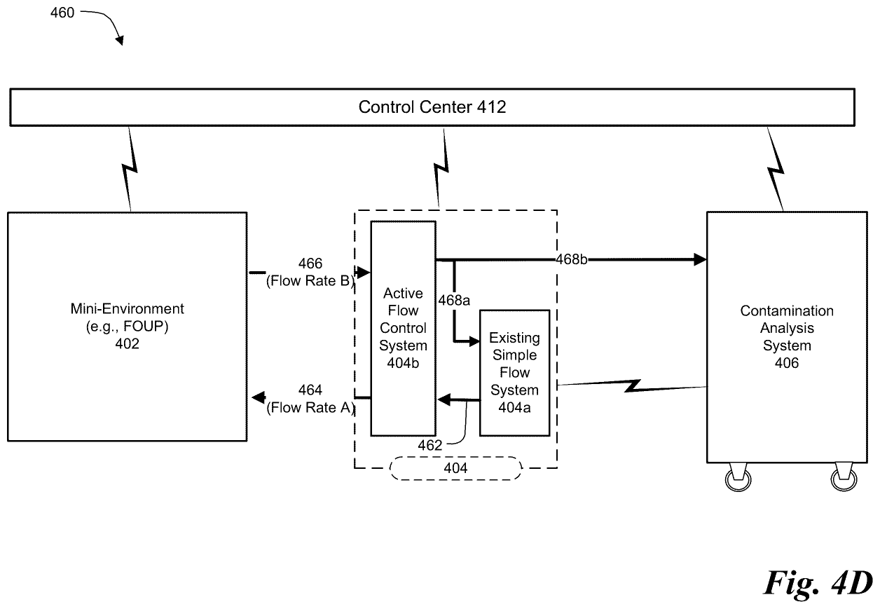

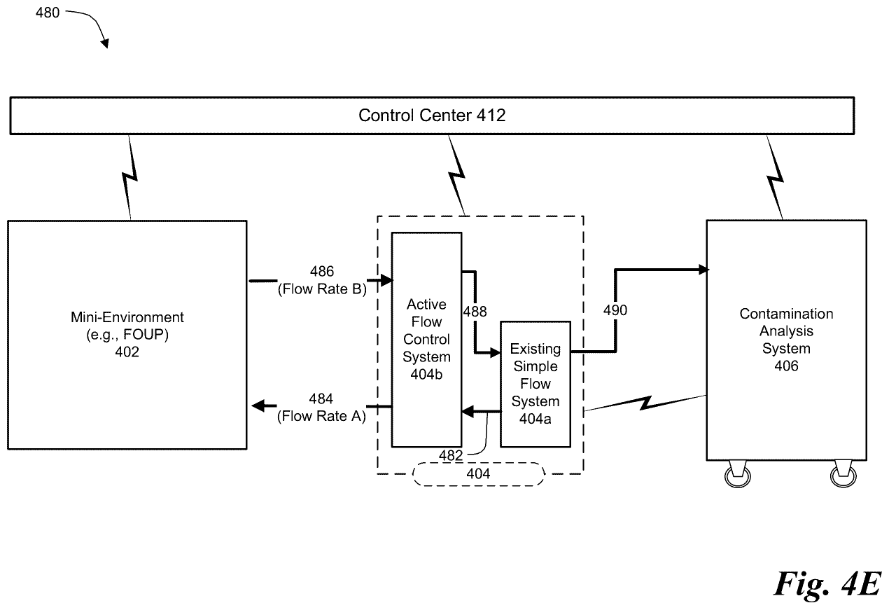

Active flow control module/system 304 can implement various different modes, can be programmable, and can be controlled locally or remotely by control center 314. The various different modes and their programmability are discussed below in connection with FIGS. 4A-4E. The active flow control system functions as a cleaning and/or flushing module/system and, in some of its modes, injects fluid into mini-environment 302 and directs samples collected from the interior of mini-environment 302 to contamination analysis system 306. Active flow control module/system 304 can be part of an existing process module, for instance the load port of a process equipment/module that the mobile carrier will be placed upon for next process step.

Contamination analysis system 306 is fluidly coupled by fluid line 312 to an outlet of active flow control system 304, so that it can be used to measure and monitor environmental conditions, for instance airborne molecular contamination (AMC), in fluid evacuated from mini-environment 302 to the contamination analysis system by the active flow control system. In the illustrated embodiment active flow control system 304 and contamination analysis system 306 are separate units, but in other embodiments flow control 304 and contamination analysis system 306 can be integrated into a single unit. In one embodiment, for instance, the active flow control system of FIG. 16 could be integrated with contamination analysis system 306, an embodiment of which is shown in FIG. 6. Programmable flow profiles can be used to obtain best efficacy on each operation mode to the targeted outcome (e.g., best sampling recovery for contamination analysis), as discussed below in connection with FIGS. 4A-4E. An embodiment of a contamination analysis system suitable to be used as contamination analysis system 306 is described below in connection with FIG. 6.

Control center 314 is communicatively coupled to mini-environment 302, active flow control system 304, and contamination analysis system 306. In addition, contamination analysis system 306 can be communicatively coupled directly to active flow control system 304. In the illustrated embodiment all communication links are wireless, but in other embodiments the communication links can be wireless, wired, or a combination of wired and wireless, and not all communication links need be of the same type. Through its communication links, control center 314 can receive data from, and transmit data to, mini-environment 302, active flow control system 304, and contamination analysis system 306. Contamination analysis system 306 can also communicate with remote control center 314 in real time, directly or through active flow control 304, to report measurement results, as shown in the figure. Contamination analysis system 306 can also communicate directly with active flow control system 304 to, among other things, regulate the flow rate through fluid line 312.

For data received at control center 314 from contamination analysis system 306, when the contaminant concentrations exceed a cleanliness criteria (which can be carried over from previous process), the control center 314 can transmit control signals to the system to instigate responsive actions-pausing the process on the sample (e.g., wafer or mask) inside the corresponding mobile carrier, for instance—to prevent contamination further to the following process equipment/module.

In an embodiment of a sampling mode or a purge mode of system 300, active flow control system 304 injects a neutral fluid at a flow rate A into mini environment 302 through fluid line 310. A neutral fluid, as that term is used herein, is a fluid or combination of fluids that is not expected to chemically react with contaminants and/or materials that are present in the mini-environment. In one embodiment a neutral fluid can be a chemically inert gas such as nitrogen (N₂), but in other embodiments it can be a gas, or a combination of gases such as clean dry air (CDA), that is not chemically inert but nonetheless is not expected to chemically react with contaminants or materials such as silicon wafer, metals, and photoresist. In the embodiments described herein the neutral fluid is a gas, but in other embodiments the neutral fluid can be a liquid or a combination of liquid and gas.

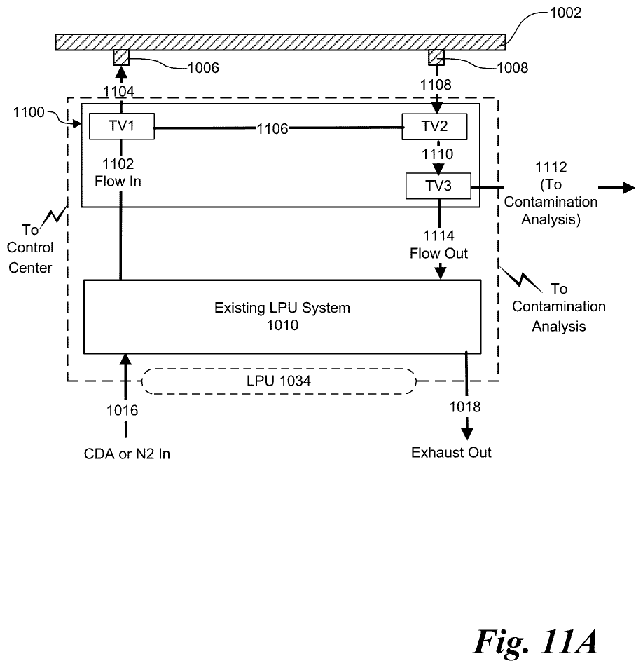

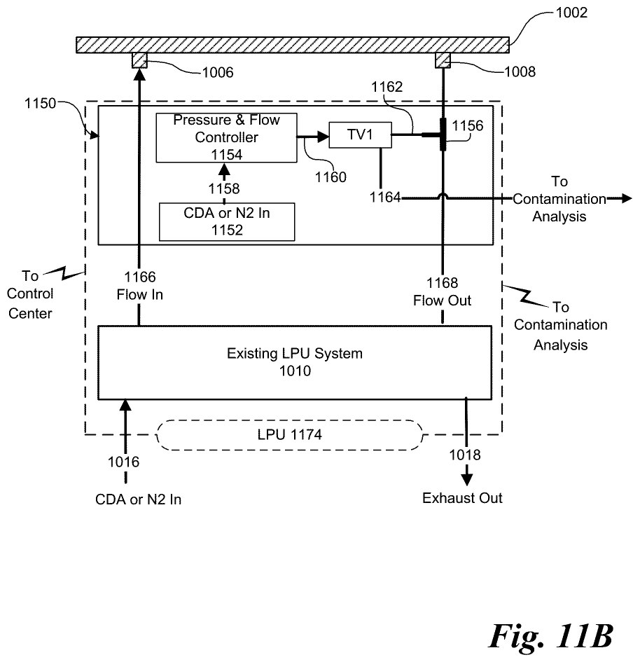

Fluid injected into mini-environment 302 exits the mini-environment through fluid line 308 at a flow rate B and is directed into the active flow control system 304. The fluid exiting through fluid line 308 is a mixed fluid—i.e., it is a mixture of the injected neutral fluid and the fluid that was in the mini-environment before injection of the neutral gas. If there were any contaminants in the mini-environment before injection of the neutral gas, they are carried out of the mini-environment in the mixed fluid. Active flow control 304 then directs some or all of the fluid entering through fluid line 308 into fluid line 312 at a flow rate C, where the fluid travels to contamination analysis system 306 for analysis. Due to fluid leakage at various places—for instance at a door through which wafers are inserted and removed from the FOUP—flow rates A, B, and C need not be the same. In one embodiment, flow rate A will be greater than flow rate B, and flow rate B will in turn be greater than flow rate C. But in other embodiments the flow rates need not follow this pattern. For instance, in an embodiment where active flow control 304 can inject additional fluid into the system (see, e.g., FIG. 11B), flow rate C can exceed one of both of flow rates A and B. In another embodiment, contamination analysis system 306 can regulate flow rate C, for instance by use of a pump (see, e.g., FIG. 6) so that flow rate C can be regulated to be different or the same as flow rates A and B. Active flow control 304 can have additional modes besides sampling and purge modes, as described below.

FIG. 3B illustrates another embodiment of an in-line measurement and monitoring system 350. System 350 is in most respects similar to system 300: it includes an LPU/active flow control system 304, a contamination analysis system 306, and a control center 314, all of which work together to analyze and monitor the environment inside a mini-environment. The primary difference between systems 300 and 350 is the type of mini-environment: in system 350, mini-environment 352 is a carrier stocker instead of a FOUP. As with FOUP 302, carrier stocker 352 has at least one inlet and at least one outlet that are coupled to an active flow control system 304. The active flow control system includes at least modes for sampling and flushing or ventilating the interior of the mini-environment. Active flow control system 304 can be part of an existing stocker setup or an external add-on system, and can be programmable and can be controlled locally or remotely by control center to perform the same functions for the stocker that is performed for a FOUP, as discussed below.

FIGS. 4A-4E illustrate further embodiments of systems for in-line contamination analysis and monitoring of conditions in a mini-environment. In the illustrated embodiments the mini-environment is a front-opening unified pod (FOUP) used to transport wafers in semiconductor fabrication, but in other embodiments the mini-environment need not be a FOUP. In some embodiments, the mini-environment can be another type of mobile wafer carrier besides a FOUP, such as but not limited to a Pod, FOUP, FOSB, PGV, Wafer cassette, Wafer SMIF, OHT, OHB, OHS, ZFS, AMR, AGV, etc. In other embodiments, the mini-environment can be a mask carrier such as, but not limited to, an EUV Pod, RSP, Reticle SMIF, Mask POD, or Mask Package. In still other embodiments the mini-environment can be a carrier stocker including, but not limited to, a stocker used for mobile carrier storage or other materials storage that require environmental monitoring and control. Some embodiments of a mini-environment can be mobile and can be moved around in a facility such as a factory, but in other embodiments they need not be mobile.

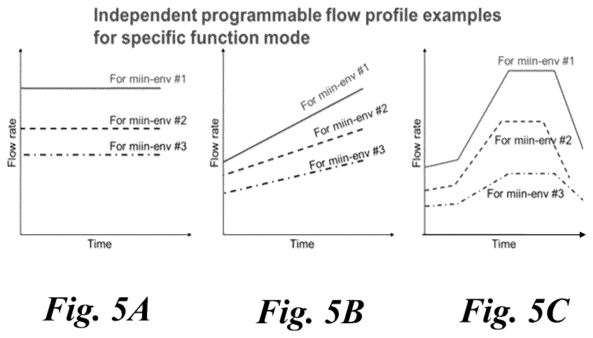

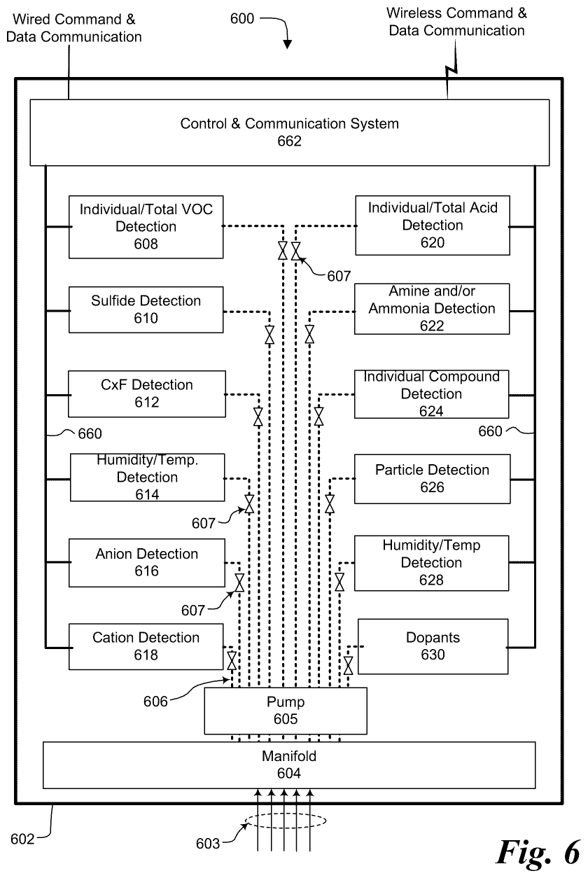

The embodiments illustrated in FIGS. 4A-4E are substantially similar to systems 300 and 350 discussed above and include similar primary components: an active flow-control system 404, a contamination analysis system 406, and a control center 412, all of which work together to analyze and monitor the environment inside a mini-environment 402. In the embodiments of FIGS. 4A-4E:
Mini-environment 402 includes at least one inlet for inflow (in liquid or gas phase) and at least one outlet for outflow (also in liquid or gas phase). The mini-environment can be any volume that is substantially closed except for its inlet and outlet. In one embodiment the number of inlets is equal to the number of outlets, but in other embodiments they need not be equal.
Load-port unit (LPU)/flow-control system 404 is coupled to the mini-environment's inlet and/or outlet to produce active flow, in liquid phase or gas phase, allowing contaminants inside the mini-environment to be carried out of the mini-environment through its outlet when the active flow is input to the inlet of mini-environment. In the embodiments of FIGS. 4A-4B, flow-control system 404 can be a complete system used as a part of mini-environment's regular ventilation/circulation flow operation/process (see, e.g., FIG. 11B). In the embodiments of FIGS. 4C-4E, the flow-control system can also be an add-on active flow-control system or adaptor module coupled between the mini-environment and an existing flow-control setup, such as an existing process module's LPU, to provide additional flow-control function without adversely affecting the mini-environment's existing operation/process (see, e.g., FIG. 11A).
The flow-control system can be designed to provide/achieve different flow profiles for the mini-environment (e.g., flow profile A, flow profile B, flow profile C, etc., as shown in FIGS. 5A-5C).
A contamination analysis apparatus 406 that is fluidly coupled to the mini-environment, either directly to its outlet port as shown in FIG. 4A or via the flow-control system, which is coupled to the outlet port of the mini-environment, as shown in FIGS. 4B-4E.
A control center 412 that is communicatively coupled by wire or wirelessly to mini-environment 402, flow-control system 404, and/or contamination analysis system 406. The control center can communicate with each element to receive data reports and information about each system's operation. The control center can also send control signals to each element to control aspects of their operation. For instance, if one or more specific contaminants measured by the contamination analysis system exceed a threshold concentration, the control center can determine a corresponding action, for example, sending an alert to user or pausing the fabrication process at the related station.
A wired or wireless communication link between contamination analysis system 406 and the flow-control system 404. This communication link allows the two to exchange data and control signals. In some embodiments, the contamination analysis system 406 can also be used to regulate the flow rate exiting the mini-environment or the flow-control system.

The embodiments illustrated in FIGS. 4A-4E have at least the above similarities but, as discussed below, they differ primarily in which active flow-control system (complete or add-on) they include and in the fluid couplings between elements.

FIG. 4A illustrates an embodiment of a system 400 that includes a mini-environment 402, a complete flow-control system 404, and a contamination analysis apparatus 406, all of which are communicatively coupled to control center 412. Contamination analysis system 406 is also communicatively coupled directly to flow-control system 404. The inlet of the mini-environment is fluidly coupled by fluid line 408 to an outlet of the flow-control system 404 and the outlet of the mini-environment is fluidly coupled by fluid line 410 to an inlet of contamination analysis apparatus 406.

FIG. 4B illustrates an embodiment of a system 420 that includes a mini-environment 402, a complete flow-control system 404, and a contamination analysis apparatus 406, all of which are communicatively coupled to control center 412. Contamination analysis system 406 is also communicatively coupled directly to flow-control system 404. An inlet of the mini-environment is fluidly coupled by fluid line 422 to an outlet of the flow-control system, an outlet of the mini-environment is fluidly coupled by fluid line 424 to an inlet of the flow-control system, and an outlet of the flow-control system is fluidly coupled by fluid line 426 to an inlet of the contaminant analysis system.

FIG. 4C illustrates an embodiment of a system 440 that includes a mini-environment 402, a load-port unit (LPU) 404 that includes an existing simple flow system 404a and an add-on active flow-control system 404b, and a contamination analysis apparatus 406, all of which are communicatively coupled to control center 412. Contamination analysis system 406 is also communicatively coupled directly to flow-control system 404. An inlet of the mini-environment is fluidly coupled by fluid line 444 to an outlet of the active flow-control system 404b and an outlet of the mini-environment is fluidly coupled by fluid line 446 to an inlet of active flow-control system 404b. Existing simple flow-control system 404a has an outlet that is fluidly coupled by fluid line 442 to another inlet of active flow-control system 404b, and another outlet of active flow-control system is fluidly coupled by fluid line 448 to an inlet of the contamination analysis system.

FIG. 4D illustrates an embodiment of a system 460 that includes a mini-environment 402, a load-port unit (LPU) 404 that includes an existing simple flow system 404a and an add-on active flow-control system 404b, and a contamination analysis apparatus 406, all of which are communicatively coupled to control center 412. Contamination analysis system 406 is also communicatively coupled directly to flow-control system 404. An inlet of the mini-environment is fluidly coupled by fluid line 464 to an outlet of the active flow-control system 404b and the outlet of the mini-environment is fluidly coupled by a fluid line 466 to an inlet of the active flow-control system. Existing simple flow-control system 404a has an outlet that is fluidly coupled by fluid line 462 to another inlet of the active flow-control system. Another outlet of active flow-control system 404b is fluidly coupled by fluid line 468a to an inlet of the existing simple flow system and by fluid line 468b to an inlet of the contaminant analysis system.

FIG. 4E illustrates an embodiment of a system 480 that includes a mini-environment 402, a load-port unit (LPU) 404 that includes an existing simple flow system 404a and an add-on active flow-control system 404b, and a contamination analysis apparatus 406, all of which are communicatively coupled to control center 412. Contamination analysis system 406 is also communicatively coupled directly to flow-control system 404. An inlet of mini-environment 402 is fluidly coupled by fluid line 484 to an outlet of the active flow-control system and an outlet of the mini-environment is fluidly coupled by fluid line 486 to an inlet of active flow-control system 404b. Existing simple flow-control system 404a has an outlet that is fluidly coupled by fluid line 482 to another inlet of the active flow-control system. Another outlet of active flow-control system 404b is fluidly coupled by fluid line 488 to an inlet of the existing simple flow system, and another outlet of the existing simple flow system is fluidly coupled by fluid line 490 to an inlet of contamination analysis system 406.

The active flow-control system—whether the integrated embodiment shown in FIGS. 3A-3B, 4A-4B, and 16 or the add-on embodiment (i.e., a flow-control system added on to an existing simple flow system) shown in FIGS. 4C-4E—can operate in one or more of the following modes:
Clean Mode. In this mode, the active flow-control system flushes a neutral fluid (such as nitrogen (N₂) or clean dry air (CDA)) through itself to clean out any contamination that can be present in the active flow-control system as a self-clean function. Contamination can be present, for instance, after running a previous sampling mode. In other embodiments, the clean mode can include additional or different functions.
Check Mode. In check mode, the active flow-control system uses the contaminant analysis apparatus to check the level of contamination within itself and/or the fluid lines to the contamination analysis apparatus as the baseline/background cleanliness, to ensure that there is no contamination within the active flow-control system itself that could affect the measurement of samples obtained from within the mini-environment using the sampling mode. In one embodiment, the check mode can be run following a clean mode to check whether the clean mode was effective in clearing existing contamination out of the active flow-control system. In another embodiment, the check mode can be run, instead or in addition, before running the sampling mode. In other embodiments, the check mode can include additional or different functions.
Sampling Mode. In sampling mode, the active flow-control system samples the interior of the mini-environment by directing an inflow of a neutral fluid (in gas or liquid phase) into the inlet of the mini-environment, and this into the interior of the mini-environment, and directing the resulting outflow from the outlet of the mini-environment to the contamination analysis apparatus, In other embodiments, the sampling mode can include additional or different functions.
Purge Mode. In purge mode, the active flow-control system directs a neutral fluid into the interior of the mini-environment to clean out any contamination that is present in the interior. In one embodiment, the purge mode can include the common purge mode and continuous purge mode of a standard load port. In other embodiments, the purge mode can include additional or different functions.

Other embodiments of the active flow-control system can include additional modes of operation in addition to those listed, and any given sequence of operations need not use all of the available modes. In the embodiments of FIGS. 4A-4E, the modes themselves, as well as their sequence, can also be programmable. The operational characteristics of each mode—e.g., its flow rate, its flow rate profile, and its duration in one embodiment—can be programmed. A particular operation includes a number of modes executed in a sequence; in one embodiment the characteristics of each mode can be programmed to be constant during a given operation, but in other embodiments the characteristics of each mode can vary during the mode and/or between occurrences of the mode within a given operation (see, e.g., FIGS. 5A-5C). Each mode, when programmable, can include functions in addition to those described above. Software, such as but not limited to the Equipment Automation Program (EAP), can be used to send commands from the control center to the LPUs to perform the different flow profiles for each mode (check mode, sampling mode, purge mode, clean mode, etc.).

In addition to the programmability of the operational characteristics of each mode, the number and types of modes executed in an operation, and their sequence, can be programmable. For example, in one embodiment a four-mode operation can include clean mode, check mode, sampling mode, and purge mode executed in that order, but a different four-mode operation can change the sequence and execute these modes in a different order. The total number of modes in a given operation can be varied—i.e., embodiments of an operation can use all the available modes or less than all the available modes. And a sequence of modes can include more than one instance of any mode. For example, an embodiment of an operation with a six-mode sequence can include more than one sampling mode, with the following sequence: clean mode, check mode, sampling mode, purge mode, sampling mode, purge mode. A sequence like this allows the system to check the effectiveness of the purge mode.

In one embodiment, a programmable sampling step can be inserted to existing flow-control system process sequence when producing active flow into the inlet of the mini-environment to obtain best recovery rate of contaminants carried from the outlet of the mini-environment to the contaminant analysis apparatus. In another embodiment, the programmable sampling flow step and flow control can be part of the add-on flow-control system/module/adaptor. The programmable sampling flow allows much flexibility in sampling and flushing/cleaning:
Different flow profiles during the inflow can be programmed dynamically for different mini-environment settings to allow/achieve effective collection and analysis to determine the original contamination concentration inside the mini-environment.
The flow rate can be fixed or can be dynamically adjusted at different flow profile during the contaminant analysis period (see, e.g., FIGS. 5A-5C).
Another flush flow step can be inserted to the flow-control system to flush/clean all the connection tubes/components inside the flow-control system and also the pipe connection to the contaminant analysis apparatus, which avoids cross-interference to next contamination measurement.
A cleanliness analysis step by the contaminant analysis apparatus to check and confirm no background contaminant interference insides the connection tubes/components of the flow-control system and connection tubes/components between output of mini-environment and the contaminant analysis apparatus before the sampling measurement step on the mini-environment.
Different flow profiles for the flush step can be programmed dynamically for different mini-environment settings.
The mini-environment can be, but is not limited to, a mobile carrier, carrier stocker, process equipment, cabinet enclosure, or stocker room for process equipment, instrument, chemical material, etc.
The flow-control system can be an existing module/system attached to the production process equipment/module or an external system that is coupled to the mini-environment, such as but not limited to a nitrogen (N₂) charger, load port flushing system, and gas ventilation system for carrier stocker, etc.
A software control sequence and/or add-on hardware modification and/or add-on module can be added to the existing flow-control system to achieve extra programmable sampling flow steps from mini-environment to the contaminant analysis apparatus for concentration measurement.

FIGS. 5A-5C illustrate embodiments of flow profiles that can be programmed in some or all of the operational modes—e.g., check mode, clean mode, sampling mode, purge mode, and other modes not listed here-available for the embodiments of FIGS. 4A-4E. The graphs illustrate profiles of flow rate with time, but in other embodiments profiles of other quantities besides flow rate can be programmed instead or in addition. FIG. 5A illustrates a constant flow-rate profile, with different constant flow rates applied to different mini-environments. FIG. 5B illustrates a linearly-increasing flow rate, again with different flow rates applied to different mini-environments. Other embodiments could also apply a non-linearly increasing profile, a linearly decreasing profile, or a non-linearly increasing or decreasing profile. FIG. 5C illustrates a more complex profile where the flow rate first increases, then is held constant, and then decreases. Again, this profile can be applied differently to different mini-environments. Generally, any arbitrary flow rate profile can be applied to a mini-environment, to different mini-environments (e.g., applying a different flow profile to a FOUP than to a carrier stocker), or to different instances of the same mini-environment (e.g., different instances of the same FOUP).

FIG. 6 illustrates an embodiment of an in-line contamination analysis system 600 that can be used in an in-line environmental monitoring system such as the ones shown in FIGS. 3A-3B and 4A-4E. System 600 is sometimes described herein as an airborne molecular contamination (AMC) system, but it is not limited to detecting only AMC. In addition to AMC, system 600 can also measure and monitor other types of contamination and other physical and chemical properties of the interior of a mini-environment. In one embodiment, system 600 can detect and measure contamination including but not limited to individual volatile organic compounds (VOCs), total VOC, individual acid, total acid, individual based, amines, particles; at the same time, the embodiment of system 600 can detect and measure other attributes like temperature, humidity, etc. The contamination analysis system can combine various analyzers or be a single instrument, and can be coupled to the mini-environments and/or flow control systems. In various embodiments:
The contaminant analysis apparatus can be connected to multiple mini-environments and/or flow control systems to monitor different mini-environment settings.
Monitoring of different mini-environments can be scheduled/controlled locally at the contaminant analysis apparatus, or remotely by the control center.
The contaminant analysis apparatus can perform self-flush cleaning flow between measurements of each mini-environment to ensure its background cleanliness with no interference to the measurement of the mini-environment.
The contaminant analysis apparatus can also perform a full connection tubes/components cleanliness analysis step to check and confirm there is no background contaminant interference before the sampling measurement step on the mini-environment.
The contamination analysis system can have a one-to-one or one-to-many correspondence with manifold piping design for connection to multiple systems, programmable for monitoring by assignment from remote control and data server (i.e., a control center as shown in FIGS. 3A-3B and 4A-4E) or from local analyzer operation.
Real-time monitoring results (e.g., test data from sampling mode or check mode) can be reported to remote control and data server (i.e., a control center as shown in FIGS. 3A-3B and 4A-4E) for decision on whether the same load port active flow control apparatus (LPU) needs further analysis or can switch to monitoring another LPU.

Contamination analysis system 600 is housed within a housing 602, which can be fixed or movable. For instance, housing 602 can be a fixed or movable cabinet. In another embodiment, contamination analysis system 600 can be integrated with a flow control system such as the one shown in FIG. 16. System 600 includes a manifold 604 with inlets that are fluidly coupled to the individual sampling tubes from a sampling tube bus 603 and an outlet that is fluidly coupled to a pump 605 that can be used, among other things, to control the flow rate into the system through the tubes in tube bus 603. Pump 605 includes one or more outlets fluidly coupled to a variety of analyzers via tubes 606. Tubes 606 include valves 607 so that output from the pump can be selectively directed to any analyzer or combination of analyzers. Tubes 606 are neutral to all AMC compounds and also do not attract AMC compounds. They can be a passivated or coated metal tube, or an inert plastic tube (e.g., PFA or Teflon) in one embodiment.

Analyzers 608-630 can include sensors or sensor arrays for their particular type of detection, but in some embodiments they can also include additional components including gas chromatographs, pre-concentrators, traps, filters, valves, and so on. Various analyzers, including chemical analyzers (VOCs, acids, bases, etc.), a particle counter, a humidity sensor, a temperature sensor, ion analyzers, dopant analyzers, etc., can be used in different embodiments. Among others, and without limitation to the analyzers listed below, embodiments of contamination analysis system 600 can include one or more of the following types of analyzers:
An analyzer to collect and analyze the concentrations of specific (individual) volatile organic compounds (VOCs), such as IPA and/or detect total concentration of VOCs.
An analyzer to collect and analyze the concentrations of specific individual acid compounds (e.g., HF, H₂SO₄, HCL, etc.) and/or detect total concentration of acids.
An analyzer to collect and analyze the concentrations of specific (individual) base compounds (e.g., NH₄OH, NaOH, etc.) and/or detect total concentration of bases.
An analyzer to collect and analyze the concentrations of specific (individual) Sulfide compounds and/or detect total concentration of Sulfides.
An analyzer to collect and analyze the concentrations of specific (individual) Amine compounds and/or ammonia (NH3) and/or detect total concentration of Amines and/or ammonia.
An analyzer that is connected to the manifold apparatus to detect air particle or aerosol counts.
An analyzer to detect sample humidity.
An analyzer to detect the sample temperature.
An analyzer to detect fluoride compounds, such as chemical coolant agents (e.g., Carbon Fluoride compounds (CxF)) or dry etching chemicals (e.g., CxFy), and/or detect total concentration of chemical cooling agents such as Carbon Fluorides or total concentration of dry etching agents.
An analyzer to collect and analyze the concentrations of specific (individual) Anions (negatively-charged ions), such as F−, Cl−, PO43-, NOx-, SO22-, and/or detect total concentration of Anions.
An analyzer to collect and analyze the concentrations of specific (individual) Cations (positively-charged ions), such as NH₄₊, and/or detect total concentration of Cations.
An analyzer to collect and analyze the concentrations of specific (individual) Metal ions and/or detect total concentration of Metal Ions.
An analyzer to collect and analyze the concentrations of specific (individual) silicon doping ions and/or detect total concentration of dopants.

Analyzers 608-628 are communicatively coupled to control and communication system 662, which integrates the operation of all the analyzers and apparatus included in apparatus 600. Control and communication system 662 is used to receive, process, and/or interpret data received from analyzers 608-628, and each analyzer and its associated valve 607 can be controlled by the control and communication system for sample analysis. In one embodiment, the hardware of control and communication system 662 can be a general-purpose computer including a processor, memory, storage, and so on, together with software having instructions that cause the listed hardware to perform the required functions. In other embodiments, however, control and communication system 662 can be a special-purpose computer such as an application specific integrated circuit (ASIC), also with software having instructions that cause it to perform the required functions.

Control and communication system 662 can be communicatively coupled, by wire or wirelessly, to one or more process equipment modules, and/or to a remote data/control server (e.g., the control center shown in FIGS. 3A-3B and 4A-4E) that gathers data from system 600 and that can control each system 600 and the process equipment to which it is fluidly and communicatively coupled. System 600 can thus receive and transmit real-time test results update or receive operation commands from the server, such as the specific sampling channel (i.e., a specific individual sampling tube) on which to perform in-line AMC analysis.

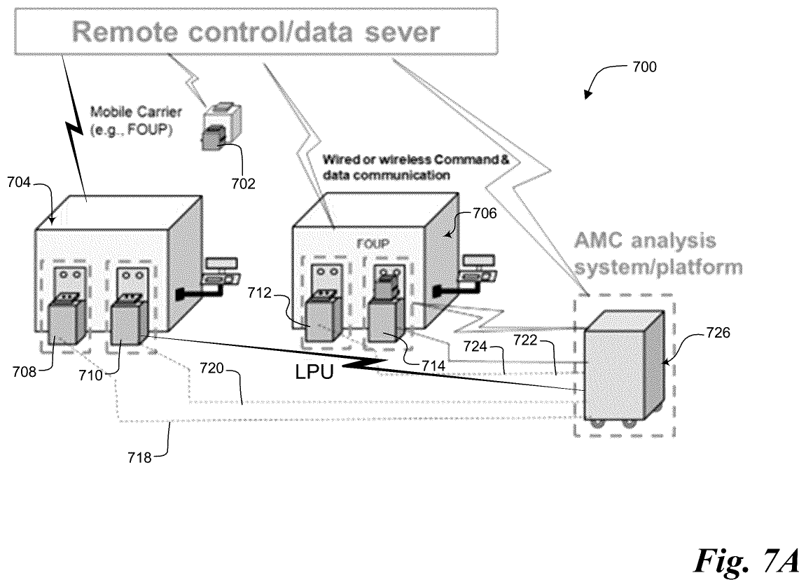

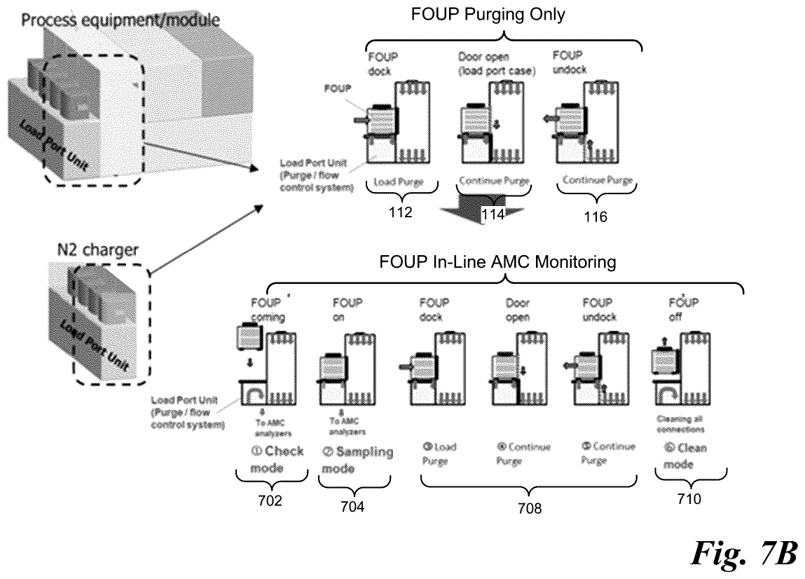

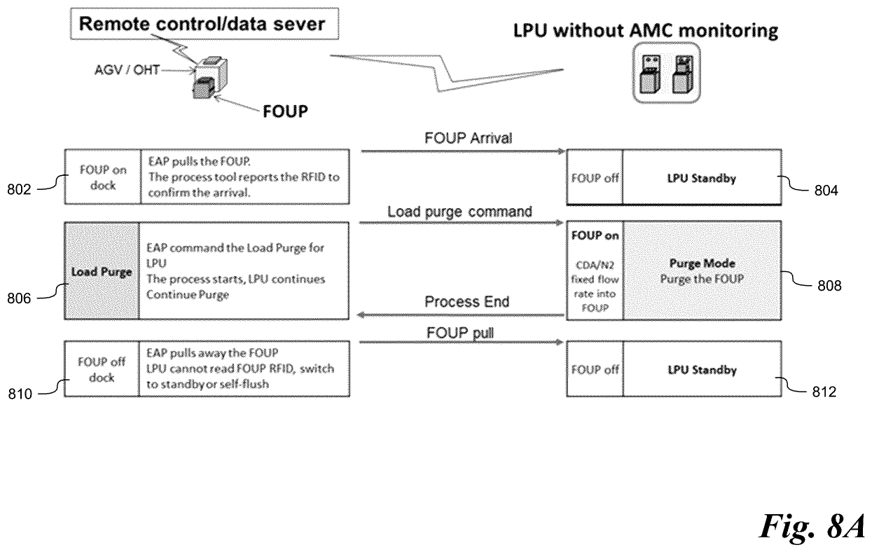

FIG. 7A illustrates an embodiment of a system and method used for in-line contamination monitoring on an existing FOUP load-port unit (LP) of process equipment and/or an N₂ charger system. FOUP 702 can be transported between different LPUs—e.g., LPUs 708 or 710 on process equipment 704 and LPUs 712 and 714 on process equipment 706—by automated guided vehicles (AGVs) or overhead transport systems (OHTs), which can be controlled by remote control/data server (e.g., the control center shown in FIGS. 3A-3B and 4A-4E). Each LPU is fluidly coupled to contamination analysis system 726 by a fluid line: LPU 708 is fluidly coupled to system 726 by fluid line 718, LPU 710 is coupled to system 726 by fluid line 720, LPU 712 is fluidly coupled to system 726 by fluid line 722, LPU 714 is fluidly coupled to system 726 by fluid line 724, and so on. In this embodiment, then, there is a many-to-one correspondence between LPUs and contaminant analysis systems—that is, multiple LPUs share a contaminant analysis system. The AMC analysis system can communicate, wirelessly or by wire, with the LPUs or control center or both for AMC monitoring command. The measurement results by AMC analyzers can be reported to an LPU, to a control center, or both, for corresponding process control. An embodiment of operation of a system where multiple LPUs share a contamination analysis system is described below in connection with FIG. 9.

FIG. 7B illustrates a comparison of an existing LPU system and an embodiment of an LPU for in-line environmental monitoring. As explained above, existing LPUs can purge the interior of the FOUP but otherwise do not measure or control contamination within the FOUP. An existing purge operation, without environmental monitoring, is shown in the top part of the figure. At step 112, movable carriers/FOUPs 104 are docked to load port 102, their bottom air inlets and outlets become fluidly coupled to mating inlets/outlets in the load port. A load port unit (LPU) housed under the FOUPs in load port 102 will then purge either nitrogen (N₂) or clean dry air (CDA) into the FOUP to flush air and/or contamination out from the FOUP's interior to an exhaust outlet under the load port, thereby keeping the mini-environment inside at a required cleanliness level. At step 114, the purge continues as the FOUP's front door is opened and the wafers inside are transferred into the other chambers of the process equipment module—front interface 106, load lock chamber 108, and process chamber 110, in that sequence—for the required fabrication steps. Purging continues while the require fabrication steps are performed, until at step 116 the wafers are returned to the interior of the FOUP, the FOUP's door is closed, and the FOUP is undocked from the load port. The CDA purge stops after the FOUP is undocked and moved to the next process equipment module.

An operation that includes in-line environmental monitoring, using a sequence of the modes described above for FIGS. 4A-4E, is shown in the top part of the figure. In the illustrated sequence, at step 702, as the FOUP is about to arrive at a load port, the LPU in the load port goes through its check mode. At step 704, after the FOUP has docked on the load port, the LPU goes through its sampling mode. After completion of the sampling mode, at step 708 the LPU goes through its purge mode, where it injects a neutral gas such as nitrogen (N₂) or clean dry air (CDA) into the FOUP to purge air and/or contamination out from the FOUP's interior to an exhaust outlet under the load port, thereby keeping the mini-environment inside at a required cleanliness level. The purge mode continues as the FOUP's front door is opened and the wafers inside are transferred into the other chambers of the process equipment module—front interface 106, load lock chamber 108, and process chamber 110, in that sequence—for the required fabrication steps. Purge mode continues while the require fabrication steps are performed, until the wafers are returned to the interior of the FOUP, the FOUP's door is closed, and the FOUP is undocked from the load port. When the FOUP has undocked from the load port, the LPU goes through its clean mode.

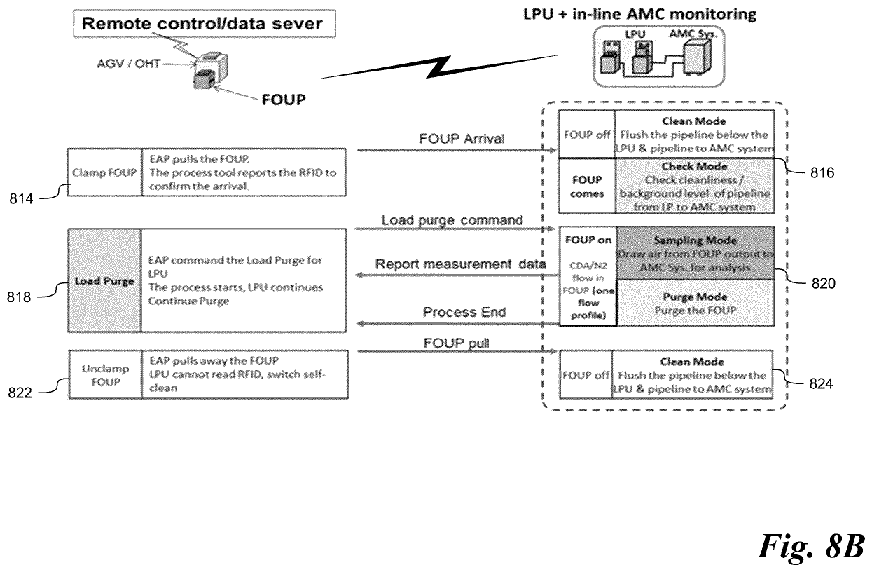

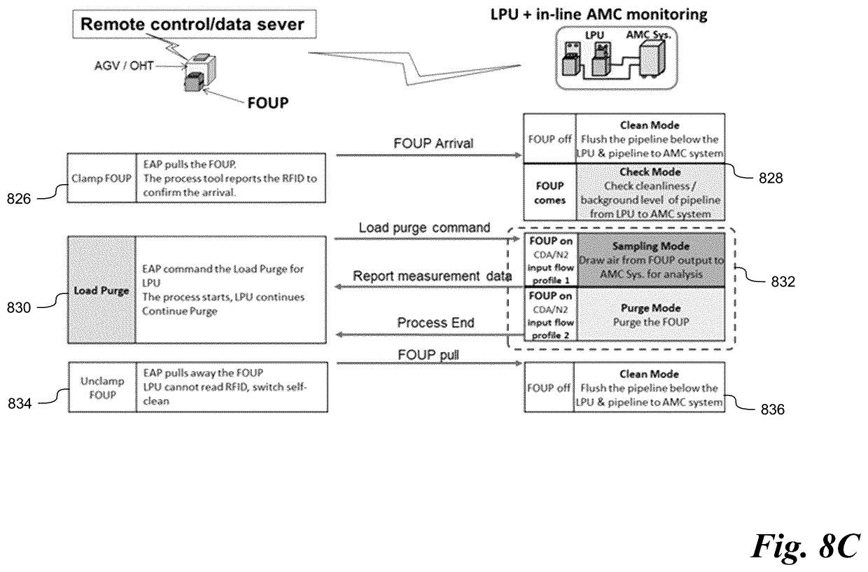

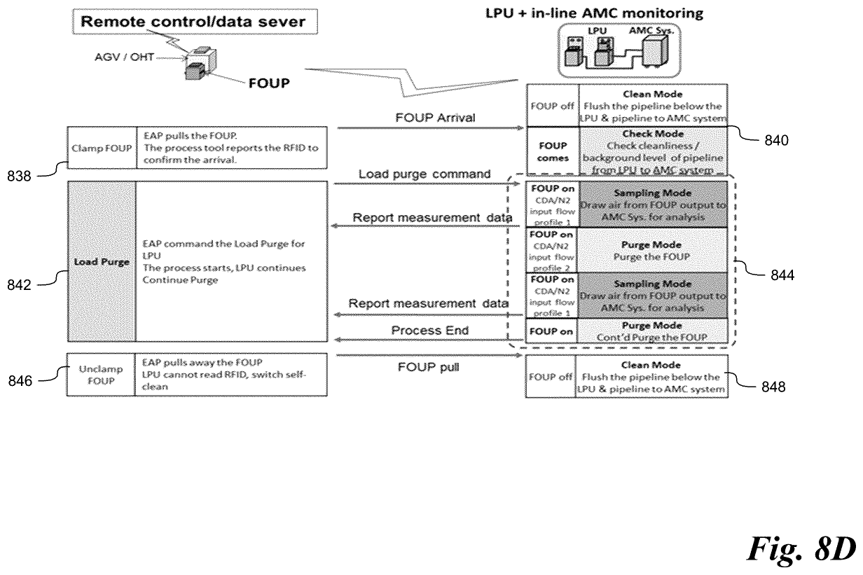

FIGS. 8A-8D illustrate embodiments of the interaction between a mini-environment and different LPU units. As in the embodiments described above, the FOUP is mobile and is carried around a facility by an overhead hoist or an automated guide vehicle (AGV), and can communicate with a remote control/date center (e.g., a control center). The control center can, among other things, know (e.g., based on a schedule) or determine the FOUP's position (e.g., based on reading an RFID tag attached to the FOUP) and communicate it other components such as the LPU. In each figure, the column on the left shows the action of the FOUP, the column on the right shows the action of the LPU, and the arrows between them illustrate their exchange of data and commands. For the embodiments of FIGS. 8B-8D, all the modes—check mode, clean mode, sampling mode, and purge mode, plus any modes not described herein—have all the characteristics, including programmability, described above.

FIG. 8A illustrates the interaction between a mini-environment, a FOUP in this embodiment, and a load-port unit (LPU) without environmental monitoring. That is, the illustrated setup can only perform a purge function when a FOUP is docked on the LPU; it does not have the functions and capability for environmental monitoring on the FOUP mini-environment's cleanliness. At block 802, the FOUP is approaching the LPU's dock, and the control center signals the FOUP's arrival to the LPU. The LPU at that stage is in a standby mode at block 804, waiting for the FOUP to dock. Once the FOUP is docked, at block 806 the control center commands the LPU to initiates a purge of the FOUP; in response to the command, at block 808 the LPU begins purging the FOUP. When the purge is complete, the LPU signals the end of the purge to the control center. At block 810, the control center signals that the FOUP should be pulled from the dock, and when the FOUP is pulled off the dock the LPU cannot sense the FOUP's presence—for instance by being unable to read an RFID tag attached to the FOUP—the process returns to its standby mode or a self-flush mode at block 812.

FIG. 8B illustrates an embodiment of the interaction between a FOUP and a load-port unit (LPU) that includes environmental monitoring of the FOUP's mini-environment. At block 814, the FOUP is approaching the LPU's dock, and the control center signals the FOUP's arrival to the LPU. The LPU at that stage, waiting for the FOUP to dock at block 816, goes through its clean mode and, before or when the FOUP is docked, goes through its check mode; clean mode and check mode are both described above. Once the FOUP is docked, at block 818 the control center commands the LPU to initiate purge mode and sampling mode; purge mode and sampling mode are also described above. in response to the command, at block 820 the LPU begins purging and sampling the FOUP. In one embodiment, sampling and purge modes can run simultaneously for at least part of the duration of the purge (e.g., sampling might only last for the first few seconds of the purge), but in another embodiment sampling and purging can run serially, with purge mode running only after completion of sampling mode.

At block 820, when sampling is in progress or complete, the LPU reports contamination measurement measurements to the control center at block 818 and then proceeds to purge mode. When purge mode is complete, the LPU report its completion to the control center at block 818. At block 822, the control center signals that the FOUP should be pulled from the dock, and when the FOUP is pulled off the dock or the LPU cannot sense the FOUP's presence—for instance because it cannot read an RFID tag attached to the FOUP—the process returns to its clean mode at block 824.

FIG. 8C illustrates another embodiment of the interaction between a FOUP and a load-port unit (LPU) that includes environmental monitoring of the FOUP's mini-environment. At block 826, the FOUP is approaching the LPU's dock, and the control center signals the FOUP's arrival to the LPU. The LPU, waiting for the FOUP to dock at block 828, goes through its clean mode and, before or after the FOUP is docked, goes through its check mode. Once the FOUP is docked, at block 830 the control center commands the LPU to initiate purge mode and sampling mode; purge mode and sampling mode are also described above. In response to the command, at block 832 the LPU begins sampling and purging the FOUP. In this embodiment, sampling and purging run serially, with purge mode running after completion of sampling mode.

At block 832, when sampling is in progress or complete, the LPU reports contamination measurements to the control center at block 830 and then proceeds to purge mode. When purge mode is complete, the LPU report completion to the control center at block 830. At block 834, the control center signals that the FOUP should be pulled from the dock, and when the FOUP is pulled off the dock or the LPU cannot sense the FOUP's presence the process returns to its clean mode at block 836.

FIG. 8D illustrates another embodiment of the interaction between a FOUP and a load-port unit (LPU) that includes environmental monitoring of the FOUP's mini-environment. At block 838, the FOUP is approaching the LPU's dock, and the control center signals the FOUP's arrival to the LPU. The LPU, waiting for the FOUP to dock at block 840, goes through its clean mode and, before or after the FOUP is docked, goes through its check mode. Once the FOUP is docked, at block 842 the control center commands the LPU to initiate purge mode and sampling mode; purge mode and sampling mode are also described above.

At block 844, in response to the command from block 842, the LPU begins sampling and purging the FOUP. In this embodiment sampling and purging run serially (i.e., one after the other), and there can be multiple sampling/purging cycles. In another embodiment, the sampling and purge modes can run simultaneously for at least part of the duration of the purge as described in FIG. 8C. The LPU runs through a first sampling mode, reports its measurement data to the control center, and then goes through a first purge mode. To check whether the first purge mode was effective, the process runs through a second sampling mode and again reports its measurements to the control center. If based on the reported measurements from the second sampling mode, the control center determines that the first purge mode was not effective, then the LPU can run through a second purge mode. In other embodiments this sampling/purge cycle can run more or less times than shown until the process determines that the purge mode has been effective in cleaning the mini-environment inside the FOUP.

At block 844, when the sampling/purging cycle is complete, the LPU reports its final purge to the control center at block 842. At block 846, the control center signals that the FOUP should be pulled from the dock, and when the FOUP is pulled off the dock or the LPU cannot sense the FOUP's presence the process returns to its clean mode at block 848.

FIG. 9 illustrates an embodiment of a monitoring schedule 900 that can be used when a single contamination analysis system monitors multiple LPUs—i.e., when there is a many-to-one correspondence between LPUs and contamination analysis system. In the illustrated embodiment there are two LPUs and one contamination analysis system, but other embodiments are not limited to these numbers (see, e.g., FIG. 7A). The contamination analysis system can be connected to multiple LPUs through a manifold system.

The disclosed embodiment includes at least the modes described above—check mode, clean mode, sampling mode, and purge mode—to achieve in-line monitoring capability for the mini-environment in a FOUP without changing the FOUP's existing process flow. In the figure, the column on the left shows the action of the first LPU, the column in the center shows the action of the contamination analysis system, and the column on the right shows the action of the second LPU. In the illustrated embodiment the same sequence of modes is applied to both LPUs, but in other embodiments different modes sequences can be applied to each LPU, for instance based on the environmental measurements received from each FOUP that docks on a particular LPU (see, e.g., FIG. 8D). All of the described modes—check mode, clean mode, sampling mode, and purge mode, etc.—have all the characteristics, including programmability, described above.

The process starts with a FOUP undocked to the first LPU, shown in the figure as LP1. With the FOUP undocked, at block 902 LP1 runs through its clean mode while the contamination analysis system remains in a standby mode at block 912. At block 904, the FOUP is about to arrive and LP1 runs its check mode and sends fluid from the check mode to the contamination analysis system at block 914. At block 906, the FOUP has docked and LP1 runs through its sampling mode and sends the sample to the contamination analysis system for analysis at block 916. After sampling mode is complete at block 906, LP1 proceeds to its purge mode at block 908. At block 910, the FOUP is undocked and LP1 runs through its self-cleaning mode.

At block 918, the contamination analysis system can optionally clean and check its own internal fluid connections while it waits for a FOUP to dock at LP2. With a FOUP undocked at block 926 LP2 runs through its clean mode. At block 928, the FOUP is about to arrive and LP2 runs its check mode and sends fluid from the check mode to the contamination analysis system, which analyzes the check-mode fluid at block 920. At block 930, the FOUP has docked and LP2 runs through its sampling mode and sends the sample to the contamination analysis system, which analyzes the check-mode fluid at block 922. After sampling mode is complete at block 930, LP2 runs through its purge mode at block 932. At block 924, the contamination analysis system can run an optional check and cleaning of its own system pipeline, and at block 934, the FOUP is undocked and LP2 runs through its self-cleaning mode.

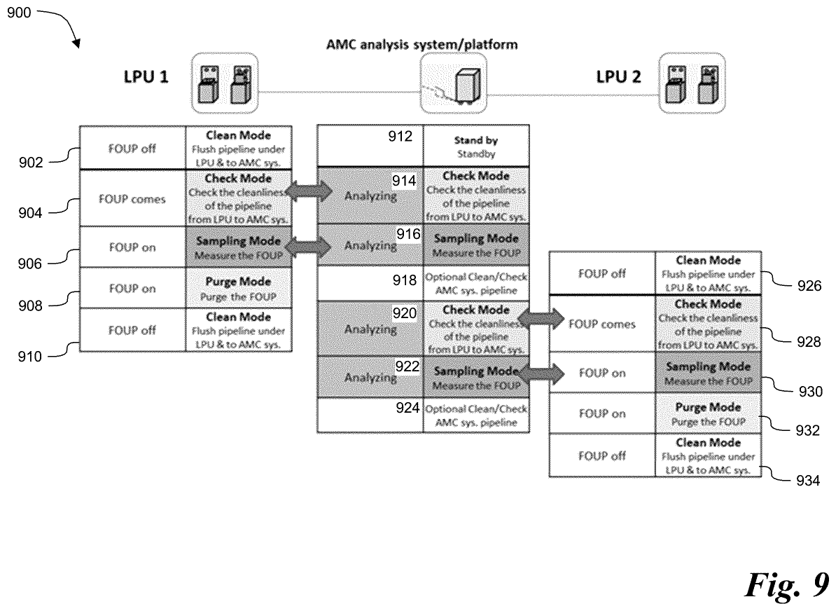

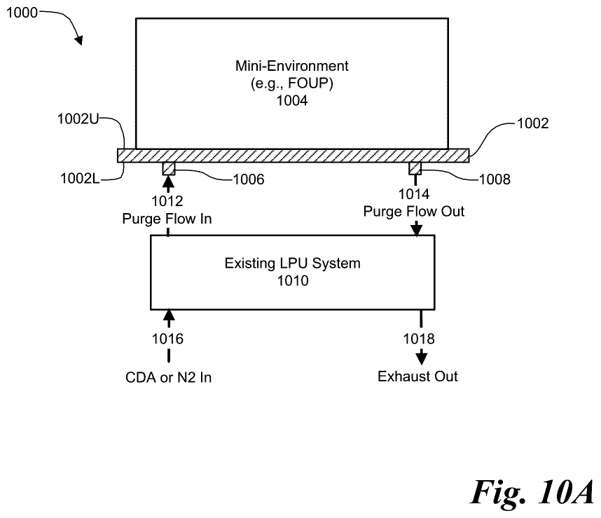

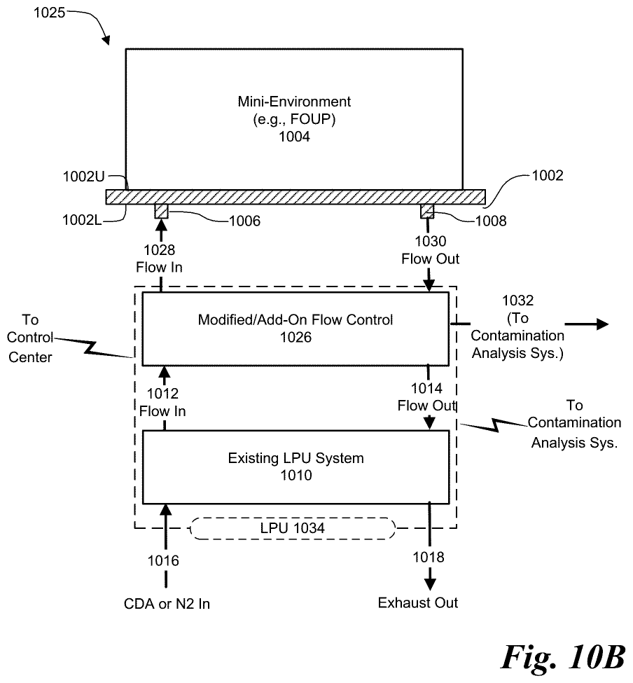

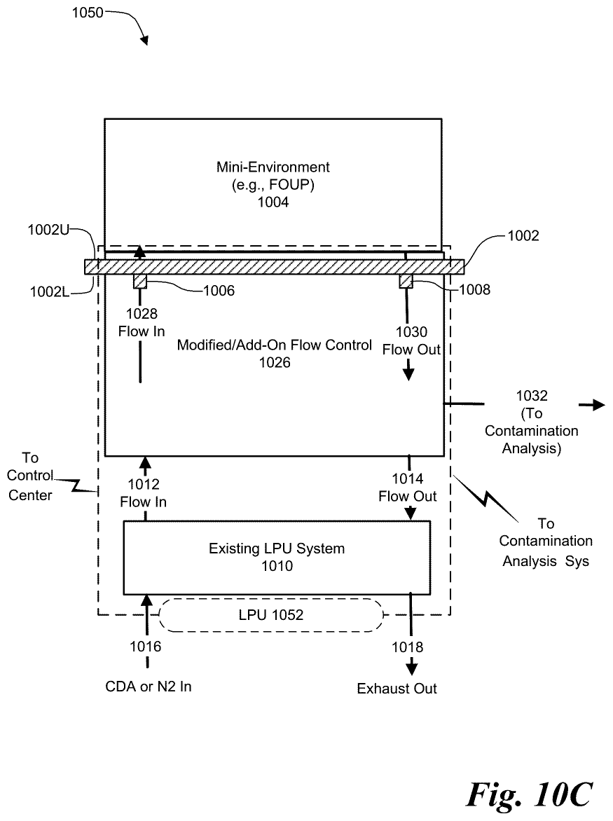

FIG. 10A illustrates an embodiment of a load port unit (LPU) 1000. In the illustrated embodiment the LPU is placed underneath a docking plate load port of a piece of process equipment (see, e.g., FIG. 1A), but in other embodiments LPU 1000 can be placed elsewhere. A load port usually includes a docking plate 1002 having an upper surface 1002U and a lower surface 1002L. Lower surface 1002L includes a fluid inlet 1006 and a fluid outlet 1008, and the interior of docking plate 1002, and its top surface 1002, include fluid connections (not shown) that fluidly couple inlet 1006 and outlet 1008 to the mini-environment inside FOUP 1004. Upper surface 1002U also includes mechanical connections to receive the FOUP 1004 and keep it removably mounted on the docking plate.

An existing LPU system 1010 has two inlets and two outlets. One outlet is fluidly coupled by fluid line 1012 to docking plate inlet 1006, and a corresponding inlet is fluidly coupled by fluid line 1014 to docking plate outlet 1008. Another inlet is fluidly coupled by fluid line 1016 to a source of neutral gas such as nitrogen (N₂) or clean dry air (CDA), and a corresponding outlet is fluidly coupled to a fluid exhaust 1018.

In operation of LPU system 1010, neutral gas is injected into the system through fluid connection 1016. The neutral gas is then injected through fluid connection 1012 and inlet 1006 into the interior of FOUP 1004 to purge the mini-environment inside the FOUP. Neutral gas injected into FOUP 1004, now also carrying contamination that was in the FOUP, then exits the FOUP through outlet 1008 and fluid line 1014 and returns to LPU system 1010. Existing LPU 1010 then exhausts the purge air, for instance to the atmosphere, through fluid line 1018. This LPU, then, is capable only of purging or flushing the mini-environment inside the FOUP.

FIG. 10B illustrates an embodiment of a load port unit (LPU) 1025. In the illustrated embodiment the LPU is placed underneath a docking plate 1002 of the load port of a piece of process equipment (see, e.g., FIG. 1), but in other embodiments LPU 1025 can be placed elsewhere. LPU system 1025 includes two main components: an existing LPU system 1010 and an add-on active flow control 1026 that together cooperate as an integrated LPU 1034. LPU 1034 is communicatively coupled to both a contamination analysis system and a control center, and the control center is also communicatively coupled to the contamination analysis system (see, e.g., FIGS. 4A-4E). In the illustrated embodiment, active flow control system 1026 adds a sampling mode, a clean mode, a check mode, and a purge mode to a purge function that already exists in the existing LPU system 1010.

In the illustrated embodiment existing LPU system 1010 and add-on flow control 1026 are separate units, but in other embodiments they can be combined into a single unit (see, e.g., FIG. 16). Existing LPU system 1010 has an outlet fluidly coupled by fluid line 1012 to an inlet of flow control 1026, and a corresponding inlet is fluidly coupled by fluid line 1014 to an outlet of flow control 1026. Another inlet of LPU system 1010 is fluidly coupled by fluid line 1016 to a source of neutral gas such as nitrogen (N₂) or clean dry air (CDA), and another outlet is fluidly coupled to a fluid exhaust 1018.

Add-on active flow control system 1026, in addition to the fluid connections to LPU system 1010 described above, has an outlet fluidly coupled by fluid line 1028 to docking plate inlet 1006, and has an inlet fluidly coupled by fluid line 1030 to docking plate outlet 1008. Add-on flow control system also has an outlet fluidly coupled by fluid line 1032 to an inlet of a contamination analysis system. The integrated LPU 1034 has communication links to the control center and to the contamination analysis system (see, e.g., FIGS. 4C-4E).

In operation of LPU system 1025, neutral gas is injected into LPU system 1010 through fluid connection 1016. The neutral gas is then injected through fluid line 1012 into add-on flow control 1026. How add-on flow control 1026 directs neutral gas entering through fluid line 1012 depends on the mode is applying—e.g., check mode, clean mode, sampling mode, or purge mode, as described above.

In check mode, flow control 1026 can direct the incoming neutral gas through fluid line 1032 to the contamination analysis system, through fluid line 1014 back to LPU system 1010, or both. In clean mode, the flow control can direct the incoming neutral gas through fluid line 1014 back to LPU system 1010. The flow control can also direct the incoming neutral fluid through fluid line 1032 to contamination analysis system to clean the fluid line 1032 and the contamination analysis system. In sampling mode, the flow control can direct the incoming neutral gas through fluid line 1028 and inlet 1006 into FOUP 1004, then direct gas exiting the FOUP through fluid line 1030 to the contamination analysis system through fluid line 1032, back through fluid line 1014 to LPU system 1010 and exhaust 1018, or both. At this stage, the fluid exiting through fluid line 1030 includes the neutral gas and any contaminants carried out of the FOUP's mini-environment by the neutral gas. In purge mode, the flow control can direct the incoming neutral gas through fluid line fluid line 1028 and inlet 1006 into FOUP 1004, then direct fluid exiting the FOUP through fluid line 1030 back through fluid line 1014 to LPU system 1010 and exhaust 1018; in this embodiment, then, flow control 1026 uses the purge function of the existing LPU system to purge the FOUP. At this stage, the fluid exiting through fluid line 1030 includes the neutral gas and any contaminants carried out of the FOUP's mini-environment by the neutral gas.

FIG. 10C illustrates an embodiment of a load port unit (LPU) 1050. LPU 1050 is in most respect similar to LPU 1025: it includes substantially the same elements, arranged and cooperate in the same way as an integrated LPU 1052, LPU 1052 is communicatively coupled to both a contamination analysis system and a control center, and the control center is also communicatively coupled to the contamination analysis system (see, e.g., FIGS. 4A-4E). In the illustrated embodiment, active flow control system 1026 adds a sampling mode, a clean mode, a check mode, and a purge mode to a purge function that already exists in the existing LPU system 1010.

The primary difference between LPUs 1025 and 1050 is the configuration of add-on flow control 1026. In some embodiments, the load port's docking plate 1002 might not provide fluid connections that enable all the functions, or enable proper operation of the modes, of add-on flow control 1026. In such cases, add-on flow control 1026 can include fluid interfaces that allow it to be fluidly coupled directly to the FOUP instead of being fluidly coupled through the docking plate. The add-on flow control's fluid interfaces can replace some or all of fluid connections of docking plate 1002—i.e., in LPU 1050 the add-on flow control can partially or fully replace the functions performed by docking plate 1002 in LPU 1025. The integrated LPU 1052 has communication links to the control center and to the contamination analysis system (see, e.g., FIGS. 4C-4E).

FIGS. 11A-11B illustrate embodiments of add-on flow control units that can be used, for instance, as the active flow-control units 404b in the embodiments shown in FIGS. 4C-4E and active flow-control units 1026 in the embodiments of FIGS. 10B-10C.

FIG. 11A illustrates an embodiment of an add-on active flow-control unit 1100. As in FIGS. 10B-10C, active flow-control unit 1100 is coupled to existing LPU system 1010, so that they cooperate as an integrated LPU 1034. Active flow-control unit 1100 includes three multi-way valves. In the illustrated embodiment they are three-way valves TV1, TV2, and TV3, but in other embodiments they need not be three-way valves but can instead by multi-way valves that are more than three-way (e.g., four-way or more). Although not shown in the drawing, all the valves can include mechanisms such as switches, motors, servos, etc., that allow them to be activated based on commands received from the control center through a communication link, as shown for instance in FIGS. 4A-4E.

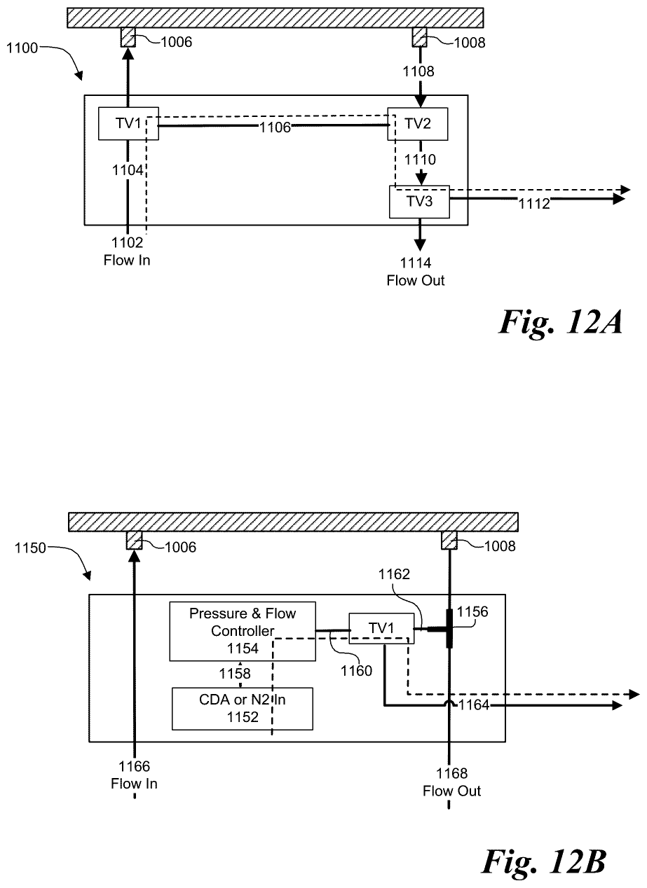

In flow-control unit 1100, three-way valves TV1-TV3 are fluidly coupled to enable the different modes discussed above. Three-way valve TV1 is fluidly coupled three fluid lines: inlet fluid line 1102, through which it can receive a neutral gas; outlet line 1104, through which it can direct the neutral gas into a mini-environment such as a FOUP through the docking plate's inlet 1006; and bypass line 1106, through which valve TV1 is fluidly coupled to valve TV2. Three-way valve TV2 is similarly fluidly coupled three fluid lines: bypass line 1106, through which it is fluidly coupled to, and can exchange fluid with, three-way valve TV1; fluid line 1108, through which it can receive fluid exiting the FOUP through the docking plate's outlet 1008; and fluid line 1110, through which valve TV2 is fluidly coupled to valve TV3. Three-way valve TV3 is also fluidly coupled three fluid lines: fluid line 1110, through which valve TV2 is fluidly coupled to valve TV3; fluid line 1112, through which it is fluidly coupled to, and can exchange fluid with, a contamination analysis system (see, e.g., FIGS. 4A-4E); and fluid line 1114, through which it can exhaust fluid exiting the FOUP through the docking plate's outlet 1008 and valve TV2. In operation of flow control unit 1100, valves TV1-TV3 can be configured to implement the modes discussed above as shown below in connection with FIGS. 12A, 13A, 14A, and 15A.

FIG. 11B illustrates an embodiment of an add-on active flow-control unit 1150. As in FIGS. 10B-10C, active flow-control unit 1150 is coupled to existing LPU system 1010, so that they cooperate as an integrated LPU 1174. Active flow-control unit 1150 includes four main components: a source of neutral gas such as nitrogen (N₂) or clean dry air (CDA) 1152; a pressure and flow controller 1154; a multi-way valve TV1; and a flow splitter 1156. Multi-way valve TV1 is shown in this embodiment as a three-way valve, but in other embodiments it can be another type of multi-way valve with more or less ports, for instance a four-way valve. Flow splitter 1156 is shown in this embodiment as a T-splitter, but in other embodiments it could be another type of flow splitter such as a Y-splitter, 4-way splitter, or some other type of splitter.

In flow-control unit 1150, neutral gas source 1152 is fluidly coupled by fluid line 1158 to pressure and flow controller 1154, and the pressure and flow controller is in turn fluidly coupled by fluid line 1160 to three-way valve TV1. Three way valve TV1 is also fluidly coupled by fluid line 1162 to flow splitter 1156, and by fluid line 1164 to a contamination analysis system. Flow splitter 1156, in addition to being fluidly coupled to valve TV1, is also fluidly coupled to fluid line 1168, which receives fluid exiting the FOUP through the outlet 1008 of docking plate 1002. Neutral fluid from an existing LPU system 1010 can also enter flow control unit 1150 through a fluid line 1166 and exit the flow control unit into the FOUP through inlet 1006 of docking plate 1002. Valve TV1 can receive fluid exiting the FOUP through outlet 1008 and flow splitter 1156, and partially or fully direct some or all of the exiting fluid through fluid line 1164 to the contamination analysis system. Although not shown in the drawing, all the elements can include mechanisms such as motors, solenoids, servos, switches, etc., that allow them to be activated based on commands received from the control center through a communication link, as shown for instance in FIGS. 4A-4E. In operation of flow control unit 1150, source 1152, controller 1154, and valve TV1 can be configured to implement the modes discussed above as shown below in connection with FIGS. 12B, 13B, 14B, and 15B.

FIG. 12A illustrates an embodiment of the operation of a check mode in flow control unit 1100. The check mode analyzes piping cleanliness to ensure no AMC background interference to the FOUP AMC measurement during next stage at sampling mode. The criteria for check mode include confirming that the cleanliness of the LPU and the contamination analysis system pipelines meet the control standards.

To implement check mode in flow control unit 1100, valve TV1 is configured to receive fluid through fluid line 1102 and direct the fluid into bypass line 1106 while preventing flow into fluid line 1104; valve TV2 is configured to receive fluid through bypass line 1106 and direct the fluid into fluid line 1110 while stopping flow through fluid line 1108; and valve TV3 is configured to receive fluid from fluid line 1110 while directing fluid into fluid line 1112. This configuration of valves results in the flow path shown by the dashed line in the figure: neutral fluid, originating for instance from existing LPU system 1010 (see FIG. 11A), enters through fluid line 1102 and travels through valve TV1 to bypass line 1106, valve TV2, fluid line 1110, valve TV3, and out through fluid line 1112 to a contamination analysis system. The fluid exiting the flow-control unit through fluid line 1112 in this check mode is a mixed fluid—i.e., it is a mixture of the injected neutral gas and the fluid that was in the flow-control unit before injection of the neutral gas. If there were any contaminants in the flow-control unit before injection of the neutral gas, they are carried out of flow control unit in the mixed fluid.

FIG. 12B illustrates an embodiment of the operation of a check mode in flow control unit 1150. To implement check mode in flow control unit 1150, source 1152 is turned on and directs neutral gas through fluid line 1158 to pressure and flow controller 1154. Pressure and flow controller 1154 directs the neutral gas through fluid line 1160 to valve TV1, which is set to direct the neutral gas only into fluid line 1164 but not into fluid line 1162. This configuration of valves results in the flow path shown by the dashed line in the figure: neutral fluid enters starting at source 1152, and travels through fluid line 1158, controller 1154, fluid line 1160, valve TV1, and finally to fluid line 1164 to a contamination analysis system. The fluid exiting the flow-control unit through fluid line 1164 in this check mode is a mixed fluid—i.e., it is a mixture of the injected neutral gas and the fluid that was in the flow-control unit before injection of the neutral gas. If there were any contaminants in the flow-control unit before injection of the neutral gas, they are carried out of flow control unit in the mixed fluid.

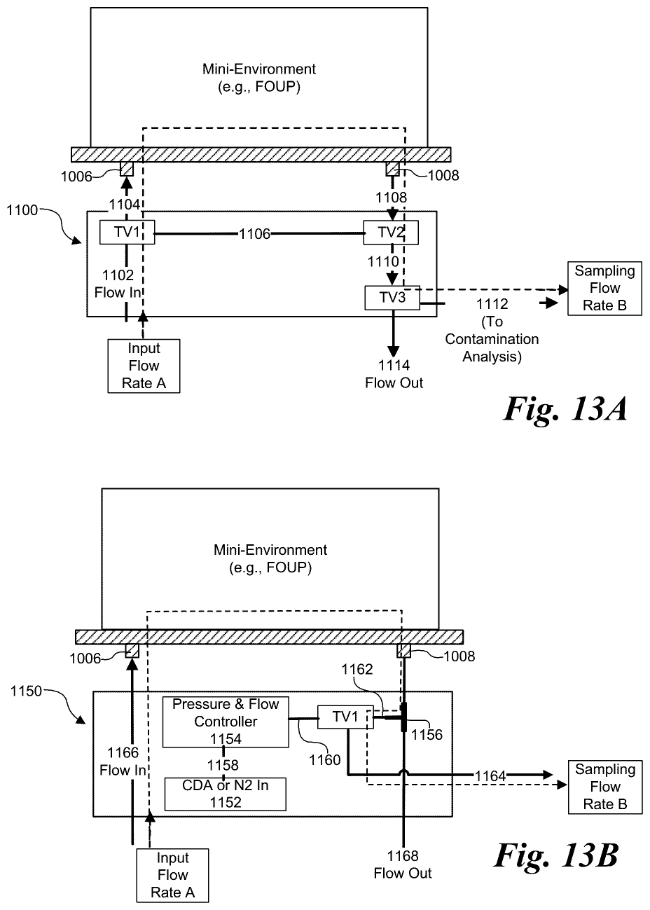

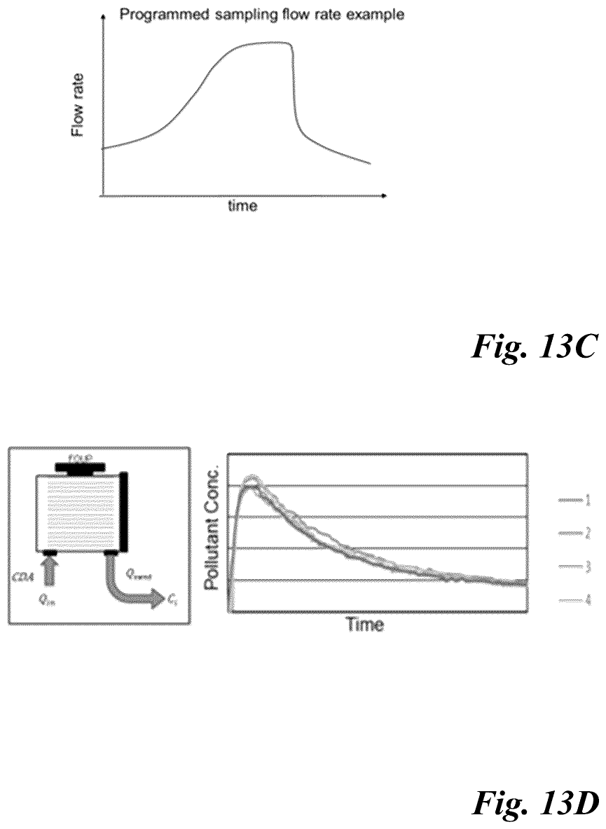

FIG. 13A illustrates an embodiment of the operation of a sampling mode in flow control unit 1100. To implement sampling mode in flow control unit 1100, valve TV1 is configured to receive fluid through fluid line 1102. Valve TV1 is set to direct the fluid into fluid line 1104 while preventing flow into bypass line 1106; valve TV2 is configured to receive fluid through fluid line 1108 and direct the fluid into fluid line 1110 while stopping flow through bypass line 1106; and valve TV3 is configured to receive fluid from fluid line 1110 and direct the fluid into fluid line 1112 but not fluid line 1114. This configuration of valves results in the flow path shown by the dashed line in the figure: neutral fluid, originating for instance from existing LPU system 1010 (see FIG. 11A), enters through fluid line 1102 at input flow rate A and travels through valve TV1 and inlet 1006 into the mini-environment inside a FOUP. The fluid then exits the FOUP through outlet 1008 and travels through fluid line 1108 to valve TV2, fluid line 1110, valve TV3, and out at flow rate B through fluid line 1112 to a contamination analysis system. The fluid exiting the mini-environment through fluid line 1108 in this sampling mode or in purge mode is a mixed fluid—i.e., it is a mixture of the injected neutral gas and the fluid that was in the mini-environment before injection of the neutral gas. If there were any contaminants in the mini-environment before injection of the neutral gas, they are carried out of the mini-environment in the mixed fluid.

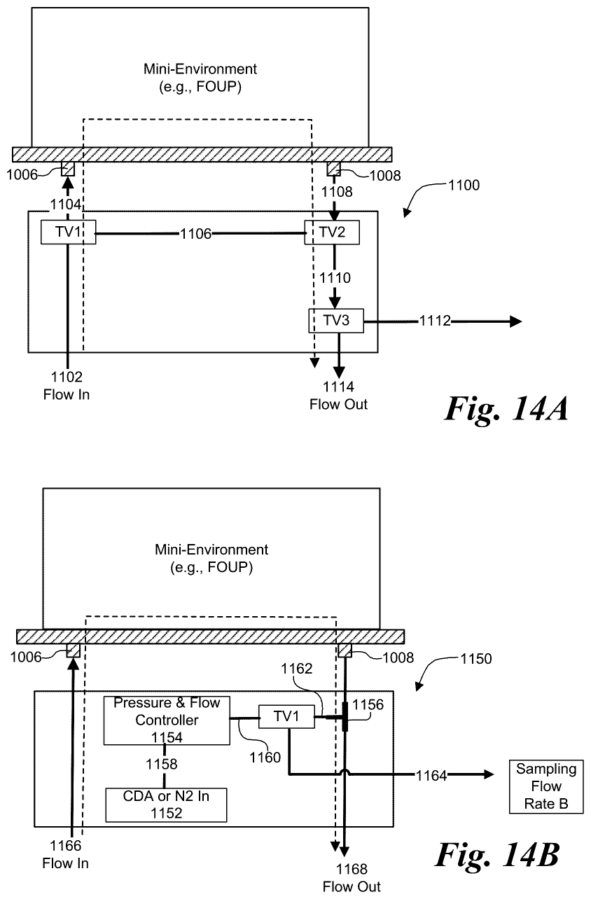

In one embodiment, flow control unit 1100 can combine the sampling mode shown in FIG. 13A with the purge mode shown in FIG. 14A. When sampling and purge modes are combined this way, flow control unit 1100 uses the purge mode already present in existing LPU system 1010 (see FIG. 11A) as part of its sampling mode, with the sampling mode running concurrently with at least an initial part of a purge mode. In one embodiment of this concurrent operation, valve TV3 can initially be set to direct some or all of the purge fluid through fluid line 1112 to the contamination analysis system, so that sampling and purge occur simultaneously. After sampling is complete, valve TV3's setting can be changed so that all the purge fluid exits through fluid line 1114 and none enters fluid line 1112.

FIG. 13B illustrates an embodiment of the operation of a sampling mode in flow control unit 1150. To implement sampling mode, valve TV1 is set to stop flow through fluid line 1160 while receive fluid through fluid line 1168 and flow splitter 1156 and directing that flow into fluid line 1164. Source 1152 and flow controller 1154 can be turned off during this mode. This configuration of valves results in the flow path shown by the dashed line in the figure: neutral fluid enters through fluid line 1166 at input flow rate A and travels into the mini-environment inside the FOUP through inlet 1006. Fluid then exits the FOUP through outlet 1008 into fluid line 1168, where flow splitter 1156 direct all or some of it into valve TV1. Valve TV1 then directs the fluid into fluid line 1164, where it travels to a contamination analysis system at a sampling flow rate B. The fluid exiting the mini-environment through fluid line 1164 in this sampling mode or in purge mode is a mixed fluid—i.e., it is a mixture of the injected neutral gas and the fluid that was in the mini-environment before injection of the neutral gas. If there were any contaminants in the mini-environment before injection of the neutral gas, they are carried out of the mini-environment in the mixed fluid. In one embodiment, flow control unit 1150 can combine the sampling mode shown in FIG. 13B with the purge mode shown in FIG. 14B. When sampling and purge modes are combined this way, flow control unit 1150 uses the purge mode already present in existing LPU system 1010 (see FIG. 11B) as part of its sampling mode, with the sampling mode running concurrently with at least an initial part of a purge mode. In one embodiment of this concurrent operation, valve TV1 can initially be set to direct some or all of the purge fluid through fluid line 1164 to the contamination analysis system, so that sampling and purge occur simultaneously. After sampling is complete, valve TV1's setting can be changed so that the purge fluid exits through fluid line 1168 and none enters fluid line 1164.

The illustrated flow path is used to perform real-time measurement on contamination purged from FOUP. The CDA injection rate into the FOUP can be optimized by controlling pressure controller and flow meter. The sampling flow rate/profile can be optimized (e.g., 5L to 10L per minute) together with injection rate to obtain the best detection response speed and recovery rate. Also, the sampling flow rate/profile can be programmed with different profiles, as illustrated in FIG. 13C. The programmable flow rate/profile recipe design not only meets the sampling requirements for targeted concentration detection (or high recovery rate), but also ensures that the gas in the FOUP can be correctly measured to provide critical contamination info to determine whether the cleanliness inside the FOUP meets the process control requirements.

FIGS. 13C-13D illustrate embodiments of the fluid inputs to, and measurement outputs from, a sampling mode. FIG. 13C illustrates a programmed flow rate profile that can be applied to the fluid input through fluid line 1102 in flow control unit 1100 or fluid line 1166 in flow control unit 1150. As explained above, any flow rate profile can be applied. But in this particular profile, the flow rate rises non-linearly with time until, at a certain time, it quickly drops. FIG. 13D illustrates an embodiment of a measurement output resulting from a sampling mode. In this embodiment, the contamination analysis system can detect multiple forms of contamination—e.g., multiple different chemical compounds and their concentrations, The detected concentrations initially increase with time, but begin to decrease with time as sampling removes more compounds from the mini-environment over time.

FIG. 14A illustrates an embodiment of the operation of a purge mode in flow control unit 1100. To implement purge mode in flow control unit 1100, valve TV1 is configured to receive fluid through fluid line 1102 and direct the fluid into fluid line 1104 while preventing flow into bypass line 1106; valve TV2 is configured to receive fluid through fluid line 1108 and direct the fluid into fluid line 1110 while preventing flow through bypass line 1106; and valve TV3 is configured to receive fluid from fluid line 1110 and direct it into fluid line 1114 while preventing flow into fluid line 1112. This configuration of valves results in the flow path shown by the dashed line in the figure: fluid enters through fluid line 1102 and travels through valve TV1, through the mini-environment inside the FOUP, to fluid line 1108, valve TV2, fluid line 1110, valve TV3, and out through fluid line 1114. In this embodiment, then, flow control unit 1100 uses the purge function already present in existing LPU system 1010 (see FIG. 11A) to implement and/or supplement or replace its own purge mode.

FIG. 14B illustrates an embodiment of the operation of a purge mode in flow control unit 1150. In one embodiment the LPU can retain the same standard purge process on FOUP during wafer process, and an optimum high purge flow rate/profile (e.g., 20L to 200L per minute) can be achieved by flow meter and pressure controller in flow control unit 1150.

To implement purge mode in flow control unit 1150, valve TV1 is set to stop flow through fluid line 1162. Source 1152 and flow controller 1154 can be turned off during this mode, so that no fluid flows to valve TV1 through fluid line 1160 or, alternatively, valve TV1 can be set to prevent flow in fluid line 1160. This configuration results in the flow path shown by the dashed line in the figure: neutral fluid enters through fluid line 1166 and travels into the mini-environment inside the FOUP through inlet 1006. Fluid then exits the FOUP through outlet 1008 into fluid line 1168, where flow splitter 1156 directs all of the flow out of the system.

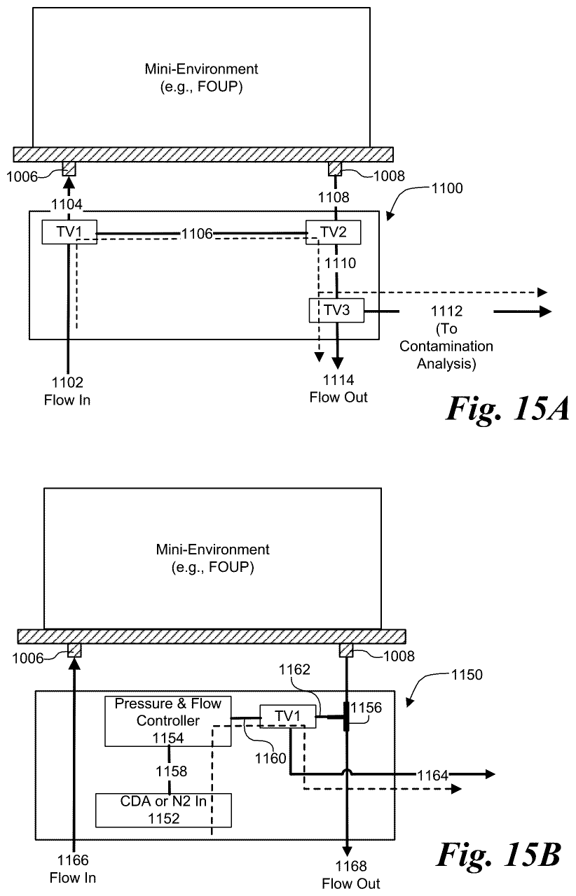

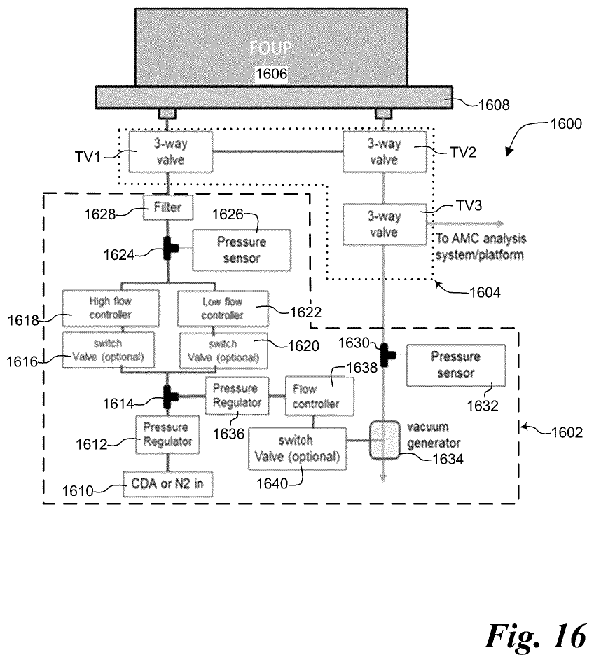

FIG. 15A illustrates an embodiment of the operation of a clean mode in flow control unit 1100. In the illustrated embodiment, after a FOUP is unloaded the system can perform the clean mode to clean all the piping within itself, as well as the piping and analyzers within the contamination analysis system, so that to ensure both are clean before the next FOUP measurement.

To implement clean mode in flow control unit 1100, valve TV1 is configured to receive fluid through fluid line 1102 and direct the fluid into bypass line 1106 while preventing flow into fluid line 1104 and valve TV2 is configured to receive fluid through bypass line 1106 and direct the fluid into fluid line 1110 while stopping flow through fluid line 1108. Valve TV3 can be configured in three different ways: it can be configured to receive fluid from fluid line 1110 and direct all the fluid into fluid line 1114; it can be configured to split the flow from fluid line 1110 into both fluid lines 1112 and fluid line 1114; or it can be configured to direct all the fluid into fluid line 1112. This configuration of valves results in the flow path shown by the dashed line in the figure: fluid enters through fluid line 1102 and travels through valve TV1 to bypass line 1106, valve TV2, fluid line 1110, valve TV3, and out through fluid line 1114 to exhaust and/or out through fluid line 1112 to a contamination analysis system. The fluid exiting the flow-control unit through fluid line 1114 in this clean mode is a mixed fluid—i.e., it is a mixture of the injected neutral gas and the fluid that was in the flow-control unit before injection of the neutral gas. If there were any contaminants in the flow-control unit before injection of the neutral gas, they are carried out of flow control unit in the mixed fluid. Directing some or all of the self-clean outlet air through fluid line 1112 not only allows flow control unit 1100 to clean both itself and the contamination analysis system, but it also allows the clean mode to be monitored while it is happening and stopped when it reaches the required level of cleanliness. If extra cleaning is desired, clean mode can also be allowed to continue after the required cleanliness level is reached.

FIG. 15B illustrates an embodiment of the operation of a clean mode in flow control unit 1150. As in the embodiment of FIG. 15A, in the illustrated embodiment the flow control unit can perform the clean mode to clean all the piping within itself, as well as the piping and analyzers within the contamination analysis system. In this embodiment, an optimum self-clean flow rate/profile is achieved by flow meter and pressure controller 1154 (e.g., 3L to 20L per minute). To implement clean mode in flow control unit 1150, valve TV1 is set to receive fluid through fluid line 1160 and direct the fluid through fluid line 1164 and to a contamination analysis system. Source 1152 and flow controller 1154 are turned on during this mode. This configuration results in the flow path shown by the dashed line in the figure: neutral fluid from source 1152 enters fluid line 1158 and travels to pressure and flow controller 1154, after which it travels through fluid line 1160 to valve TV1. Valve TV1 then directs the flow to a contamination analysis system through fluid line 1164. The fluid exiting the flow-control unit through fluid line 1164 in this clean mode is a mixed fluid—i.e., it is a mixture of the injected neutral gas and the fluid that was in the flow-control unit before injection of the neutral gas. If there were any contaminants in the flow-control unit before injection of the neutral gas, they are carried out of flow control unit in the mixed fluid. As in the embodiment of FIG. 15A, directing some or all of the self-clean outlet air through fluid line 1164 not only allows flow control unit 1150 to clean both itself and the contamination analysis system, but it also allows the clean mode to be monitored while it is happening and stopped when it reaches the required level of cleanliness. If extra cleaning is desired, clean mode can also be allowed to continue after the required cleanliness level is reached.

FIG. 16 illustrates an embodiment of an integrated load port unit (LPU) 1600. LPU 1600 includes a load port unit 1602 fluidly coupled to an active flow control 1604; it is thus referred to as “integrated” because it combines the functions of a regular load port unit 1602 with the functions of an add-on active flow control 1604 in a single unit, as shown for instance in FIGS. 4A-4B.

Load port unit 1602 includes a fluid delivery part through which it delivers fluid to active flow control 1604 and, through the active flow control, to a mini-environment such as a FOUP 1606 fluidly coupled to a load port docking plate 1608. The fluid delivery part starts with a neutral gas source 1610, which can deliver nitrogen (N₂) or clean dry air (CDA). A pressure regulator 1612 is fluidly coupled the outlet of source 1610, and a flow splitter 1614 is fluidly coupled to the outlet of the pressure regulator. One outlet of flow splitter 1614 is coupled to a flow controller that include parallel high-flow and low-flow branches. The high-flow and low-flow branches are arranged in parallel so that each can be used independently to supply a larger range of flow rates (i.e., higher and lower) than could be supplied by a single flow controller. The high-flow branch includes a switch valve 1616 fluidly coupled to an outlet of flow splitter 1614 and a high-flow controller 1618 fluidly coupled to an outlet of switch valve 1616. The low-flow branch includes a switch valve 1620 fluidly coupled to an outlet of flow splitter 1614 and a low-flow controller 1622 fluidly coupled to an outlet of switch valve 1620. Another optional flow splitter 1624 has its inlet coupled to an outlet of the flow controller. An optional pressure sensor 1626 is fluidly coupled to one outlet of flow splitter 1624, and an optional filter 1628 is fluidly coupled to another outlet of the flow splitter. Filter 1628 is then fluidly coupled to valve TV1 in active flow control 1604.

Load port unit 1602 also includes a fluid receiving part through which it can receive fluid from active flow control 1604. The fluid receiving part includes a flow splitter 1630 whose inlet is fluidly coupled to an outlet of valve TV3 of active flow control 1604. The outlets of flow splitter 1630 can be fluidly coupled to a pressure sensor 1632 and a vacuum generator 1634. The fluid delivery and receiving parts of load port unit 1602 are fluidly coupled to each other by a pressure regulator 1636 whose inlet is coupled to an outlet of flow splitter 1614, a flow controller 1638 whose inlet is fluidly coupled to an outlet of pressure regulator 1636, and an optional switch valve 1640 whose inlet is fluidly coupled to an outlet of flow controller 1638 and whose outlet is fluidly coupled to vacuum generator 1634.

Active flow control 1604 is fluidly coupled to an outlet and inlet of load port unit 1602: valve TV1 is coupled to the outlet of filter 1628 and flow splitter 1630 is coupled to an outlet of valve TV3. In the illustrated embodiment, active flow control 1604 is recognizable as add-on active flow control 1100, shown in FIG. 11A, and can operate in any of the modes described for that flow control. In other embodiments, active flow control 1604 could use active flow control 1150, shown in FIG. 11B, and can similarly operate in any of the modes described for that flow control. Because active flow control 1604 and its operation are described in detail above, the description is not repeated here.

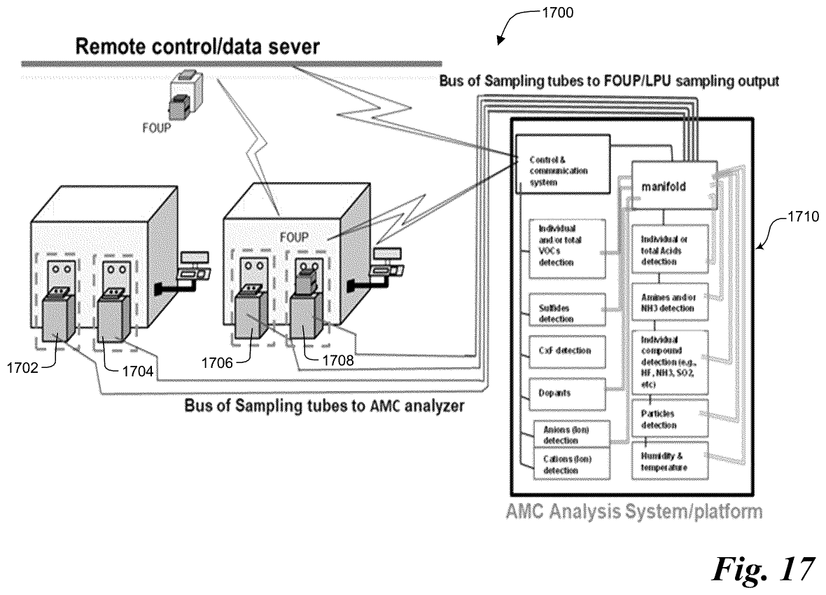

FIG. 17 illustrates an embodiment of a load-port based in-line contamination monitoring system 1700 for mobile mini-environments. The illustrated embodiment can be used together with the LPU embodiments described above. System 1700 shares many similarities with system 700: it includes multiple LPUs 1702-1708, each coupled by a fluid line to a single contamination analysis system 1710, so that there is a many-to-one correspondence between LPUs and contamination analysis systems. An embodiment of a contamination analysis system is described above in connection with FIG. 6. In this or other embodiments of system 1700:
The contamination analysis system can be one-to-one or one-to-many with manifold piping design for connection to multiple LPUs, and can be programmable for monitoring by assignment from remote control and data server or from local analyzer operation.
Real-time monitoring results (e.g. test data from sampling, check mode) is reported to remote control and data server for decision on whether an LPU needs further analysis or the system should switch to another LPU monitoring.
The contamination analysis system can be connected to multiple mini-environments and/or flow control systems to monitor different mini-environment settings.
The monitoring of different mini-environments can be scheduled/controlled locally at the contaminant analysis apparatus or by the control center remotely.
The contamination analysis system can perform self-cleaning flow between measurements of each mini-environment to ensure its background cleanliness with no interference to the measurement on the mini-environment.
The contamination analysis system can also perform a full connection tubes/components cleanliness analysis step to check and confirm no background contaminant interference before the sampling measurement step on the mini-environment.
The contamination analysis system can be one-to-one or one-to-many with manifold piping design for connection to multiple systems, programmable for monitoring by assignment from remote control & data server or from local analyzer operation.
The contamination analysis system can communicate wirelessly or by wire with either or both control center and LPU for contamination monitoring on a targeted FOUP. Real-time monitoring results (e.g. test data from sampling, check mode) is reported to remote control & data server for decision on whether the same LPU needs further analysis or switch to another LPU monitoring.

The above description of embodiments is not intended to be exhaustive or to limit the invention to the described forms. Specific embodiments of, and examples for, the invention are described herein for illustrative purposes, but various modifications are possible.

What is claimed is:

1. An apparatus comprising:
an active flow control system including one or more inlets and one or more outlets, wherein:
a first outlet of the one or more outlets is configured to be fluidly coupled to an inlet of a mini-environment, and
a first inlet of the one or more inlets is configured to be coupled to an outlet of the mini-environment; and
wherein the active flow control system includes one or more flow control configurations, wherein each flow control configuration corresponds to a flow control modes, the one or more flow configurations including a configuration corresponding to a sampling mode comprising:
injecting a neutral fluid from the first outlet into the inlet of the mini-environment, and
directing a mixed fluid exiting through the outlet of the mini-environment to the first inlet of the active flow control system, the mixed fluid being a combination of the neutral fluid and the fluid that was in the mini-environment before injecting the neutral fluid into the mini-environment.

2. The apparatus of claim 1 wherein the active flow control system can be coupled to one or multiple mini-environments.

3. The apparatus of claim 1, further comprising a control center communicatively coupled to the active flow control system, wherein the control center can transmit instructions to the active flow control system that, when executed, cause the active flow control system to execute one or more of the flow control modes.

4. The apparatus of claim 3, further comprising a contamination analysis system adapted to be communicatively coupled to the control center and fluidly coupled to a second outlet of the one or more outlets of the active flow control system.

5. The apparatus of claim 4 wherein the contamination analysis system is also communicatively coupled directly to the active flow control system.

6. The apparatus of claim 4 wherein the instructions include instructions that, when executed, cause the active flow control system to direct the mixed fluid to the contamination analysis system.

7. The apparatus of claim 4 wherein the contamination analysis system can be coupled to one or multiple active flow control systems.

8. The apparatus of claim 4 wherein the one or more flow control modes include:
a check mode in which the active flow control system checks itself for contamination,
a clean mode in which the active flow control system cleans itself of contamination, and
a purge mode in which the active flow control system purges contamination from the mini-environment.

9. The apparatus of claim 8 wherein the active flow control system is fluidly coupled to an existing load port unit with an existing purge function.

10. The apparatus of claim 9 wherein the purge mode uses or supplements or replaces the purge function of the existing load port unit.

11. The apparatus of claim 8 wherein the active flow control system adds the sampling mode, the clean mode, and the check mode to the existing purge function.

12. The apparatus of claim 11 wherein the sampling mode and the purge mode run concurrently for at least an initial part of the purge mode.

13. The apparatus of claim 8 wherein, in addition to cleaning the active flow control system, the clean mode cleans the contamination analysis system.

14. The apparatus of claim 8 wherein each flow control mode is programmable.

15. The apparatus of claim 14 wherein each flow control mode can be programmed to use a flow rate profile and the flow profile can be adjustable.

16. The apparatus of claim 15 wherein different flow rate profiles can be used in different flow control modes for specific mini-environments.

17. The apparatus of claim 16 wherein one or more new flow control modes can be added to existing flow control modes.

18. The apparatus of claim 3 wherein the instructions include instructions that, when executed, cause the active flow control system to execute an operation, the operation including multiple flow control modes selected from the one or more flow control modes and executed in a sequence.

19. The apparatus of claim 18 wherein the sequence of flow control modes in the operation is changeable and programmable.

20. The apparatus of claim 18 wherein the sequence of flow control modes can include multiple instances of any flow control mode.

21. The apparatus of claim 1 wherein the first inlet and the first outlet of the active flow control system are fluidly coupled to a load port of a process module.

22. The apparatus of claim 21 wherein a clean mode, a check mode, and a sampling mode are added to existing load port's purge mode.

23. The apparatus of claim 1 wherein the active flow control system is an add-on adaptor module coupled to an existing simple flow control system.
